(19) United States
(12) Patent Application Publication
(10) Pub. No.: US 2021/0169397 A1
(43) Pub. Date: Jun. 10, 2021

(54) IMAGE PROCESSING APPARATUS, IMAGE PROCESSING METHOD, AND NON-TRANSITORY COMPUTER-READABLE MEDIUM

(71) Applicants: CANON KABUSHIKI KAISHA, Tokyo (JP); KEIO UNIVERSITY, Tokyo (JP)

(72) Inventors: Hiroki Kajita, Tokyo (JP); Nobuaki Imanishi, Tokyo (JP); Sadakazu Aiso, Tokyo (JP); Moemi Urano, Tokyo (JP); Kenichi Nagae, Kanagawa (JP); Kazuhito Oka, Tokyo (JP)

(21) Appl. No.: 17/179,446

(22) Filed: Feb. 19, 2021

Related U.S. Application Data
(63) Continuation of application No. PCT/JP2019/032586, filed on Aug. 21, 2019.

(30) Foreign Application Priority Data
Aug. 24, 2018 (JP) 2018-157752
Aug. 24, 2018 (JP) 2018-157755
Aug. 24, 2018 (JP) 2018-157785

(51) Int. Cl. A61B 5/00; A61B 5/107

(52) U.S. Cl. CPC A61B 5/418; A61B 5/0095; A61B 5/748; A61B 5/1075; A61B 5/1079; A61B 5/742

(57) ABSTRACT

An image processing apparatus includes a data acquisition unit configured to acquire, in time series, first image data that have been generated based on acoustic waves generated by irradiating a subject, into which a contrast agent has been injected, with light a plurality of times and that correspond respectively to the plurality of times of light irradiation; and an image generation unit configured to generate second image data indicating a region corresponding to the contrast agent in the plurality of first image data on the basis of the plurality of the first image data acquired in time series.

### CROSS-REFERENCE TO RELATED APPLICATIONS

This application is a Continuation of International Patent Application No. PCT/JP2019/032586 filed Aug. 21, 2019, which claims the benefit of Japanese Patent Application No. 2018-157752 filed Aug. 24, 2018, Japanese Patent Application No. 2018-157755 filed Aug. 24, 2018 and Japanese Patent Application No. 2018-157785 filed Aug. 24, 2018, all of which are hereby incorporated by reference herein in their entirety.

### BACKGROUND OF THE INVENTION

### Field of the Invention

The present invention relates to image processing on an image generated by photoacoustic imaging.

### Description of the Related Art

In the examination of blood vessels, lymphatic vessels, and the like, photoacoustic imaging (also called “photoultrasonic imaging”) using a contrast agent is known. PTL 1 describes a photoacoustic image generator that uses a contrast agent, which is utilized for contrasting, for example, lymphatic nodes and lymphatic vessels, as an evaluation target, and emits light having a wavelength that the contrast agent absorbs to generate a photoacoustic wave.

However, in the photoacoustic imaging described in Patent Literature 1, it may be difficult to ascertain the structure of a contrast-enhanced object inside a subject (for example, running of blood vessels, lymphatic vessels, and the like). In addition, it is considered difficult to ascertain the state of the structure. Furthermore, it could be inconvenient for a user to observe the structure.

An object of the present invention is to provide an image processing apparatus utilized for a system that facilitates ascertaining the structure and state of a contrast-enhanced object by photoacoustic imaging and that improves convenience in observing the structure of the contrast-enhanced object.

### CITATION LIST

### Patent Literature

PTL 1 International Publication Pamphlet No. WO 2017/002337

### SUMMARY OF THE INVENTION

One aspect of the present invention for solving the above problems is an image processing apparatus including: a data acquisition unit configured to acquire, in time series, first image data that have been generated based on acoustic waves generated by irradiating a subject, into which a contrast agent has been injected, with light a plurality of times and that correspond respectively to the plurality of times of light irradiation; and an image generation unit configured to generate second image data indicating a region corresponding to the contrast agent in the plurality of first image data on the basis of the plurality of the first image data acquired in time series.

Further, another aspect of the present invention is an image processing apparatus processing image data generated based on photoacoustic waves generated from inside a subject by irradiating the subject with light, the image processing apparatus including: a state estimation unit configured to estimate a state of a lymphatic vessel by image analysis of the image data including a region of the lymphatic vessel in the subject.

Further, another aspect of the present invention is an image processing apparatus processing image data generated based on photoacoustic waves generated from inside a subject by irradiating the subject with light, the image processing apparatus including: a display control unit configured to display the image data and an input interface that receives an input related to a region of interest, which is a part of a region of a lymphatic vessel in the subject among the image data, on a display device; and a storage control unit configured to store the image data in association with information inputted via the input interface.

Further features of the present invention will become apparent from the following description of exemplary embodiments with reference to the attached drawings.

### BRIEF DESCRIPTION OF THE DRAWINGS

FIG. 1 is a block diagram of a system according to a first embodiment.

FIG. 2 is a block diagram showing an image processing apparatus and a peripheral configuration according to the first embodiment.

FIG. 3 is a detailed block diagram of a photoacoustic device according to the first embodiment.

FIG. 4 is a schematic diagram of a probe according to the first embodiment.

FIG. 5 is a flowchart of an image processing method performed by the system according to the first embodiment.

FIG. 6 is a spectrum diagram showing the relationship between the concentration of ICG and an absorption coefficient.

FIGS. 7A to 7D are graphs showing the calculated values of a formula (1) for each wavelength and the concentration of a contrast agent.

FIG. 8 is a diagram showing the relationship between the concentration of ICG and the calculated values of the formula (1).

FIG. 9 is a molar absorption coefficient spectrum diagram of oxyhemoglobin and deoxyhemoglobin.

FIG. 10 is a diagram showing a GUI displayed in the first embodiment.

FIGS. 11A and 11B are diagrams illustrating a process of extracting a region corresponding to a contrast agent.

FIG. 12 is a diagram illustrating a process of extracting a region corresponding to a contrast agent.

FIGS. 13A and 13B are photoacoustic images of the right forearm extension side when the concentration of ICG is changed.

FIGS. 14A and 14B are photoacoustic images of the left forearm extension side when the concentration of ICG is changed.

FIGS. 15A and 15B are photoacoustic images of the inside of the left and right lower legs when the concentration of ICG is changed.

FIG. 16 is a flowchart of an image processing method according to a third embodiment.

FIG. 17 is a flowchart of a process for displaying the classification result of lymphatic vessels.

FIG. 18 is a diagram illustrating a spectral image of a subject.

FIG. 19 is a diagram illustrating classification according to the state of a lymphatic vessel.

FIG. 20 is a diagram illustrating the classification of lymphatic vessels according to the abundance, area ratio, and volume ratio.

FIG. 21 is a diagram illustrating the classification of lymphatic vessels according to the distance from veins.

FIG. 22 is a flowchart of an image processing method according to a fourth embodiment.

FIG. 23 is a diagram illustrating a GUI according to the fourth embodiment.

FIG. 24 is a diagram showing a display example of the classification result of lymphatic vessels according to the fourth embodiment.

### DESCRIPTION OF THE EMBODIMENTS

The preferred embodiments of the present invention will be described below with reference to the drawings. However, the dimensions, materials, shapes, and relative arrangements of the components described below should be changed, as appropriate, depending on the configuration of the device to which the invention is applied and various conditions. Therefore, these are not intended to limit the scope of the present invention to the following description.

The photoacoustic image obtained by the system according to the present invention reflects the amount and rate of absorption of light energy. The photoacoustic image represents the spatial distribution of at least one type of subject information such as the generated sound pressure (initial sound pressure) of the photoacoustic wave, the light absorption energy density, and the light absorption coefficient. The photoacoustic image may be an image representing a two-dimensional spatial distribution or an image (volume data) representing a three-dimensional spatial distribution. The system according to the present embodiment generates a photoacoustic image by capturing an image of a subject into which a contrast agent has been injected. In order to ascertain the three-dimensional structure of the contrast-enhanced object, the photoacoustic image may represent an image representing a two-dimensional spatial distribution in the depth direction from the surface of the subject or may represent a three-dimensional spatial distribution.

Further, the system according to the present invention can generate a spectral image of a subject using a plurality of photoacoustic images corresponding to a plurality of wavelengths. The spectral image is generated by using photoacoustic signals corresponding to each of the plurality of wavelengths and based on the photoacoustic waves generated by irradiating the subject with light of a plurality of different wavelengths.

The spectral image may show the concentration of a specific substance in the subject, which is generated by using the photoacoustic signals corresponding to each of the plurality of wavelengths. When the light absorption coefficient spectrum of the contrast agent used and the light absorption coefficient spectrum of the specific substance are different, the image value of the contrast agent in the spectral image and the image value of the specific substance in the spectral image are different. Therefore, the region of the contrast agent and the region of the specific substance can be distinguished from each other according to the image value of the spectral image. The specific substance is a substance constituting the subject, such as hemoglobin, glucose, collagen, melanin, fat and water. Also, in this case, a contrast agent having a light absorption spectrum different from the light absorption coefficient spectrum of the specific substance is selected. Further, the spectral image may be calculated by a different calculation method depending on the type of the specific substance.

In the embodiments described below, a spectral image having an image value calculated by using the calculation formula (1) for oxygen saturation degree will be described. The present inventors have found that where a measured value I(r) of the photoacoustic signal obtained with a contrast agent for which the wavelength dependence of the absorption coefficient shows a tendency that is different from that of oxyhemoglobin and deoxyhemoglobin is substituted in the formula (1) for calculating the oxygen saturation degree of blood hemoglobin (which may be an index having a correlation with the oxygen saturation degree) on the basis of photoacoustic signals corresponding to each of a plurality of wavelengths, a calculated value Is(r) greatly deviates from the possible numerical range of the oxygen saturation degree of hemoglobin. Therefore, where a spectral image having this calculated value Is(r) as an image value is generated, it becomes easy to separate (distinguish) the hemoglobin region (blood vessel region) and the region where the contrast agent is present (for example, the region of a lymphatic vessel when the contrast agent is injected into the lymphatic vessel) inside the subject on the image.

$$[ Math . 1 ]$$

$$Is ( r ) = \frac{\frac{{I}^{{λ}_{2}} ( r )}{{I}^{{λ}_{1}} ( r )} \cdot {ɛ}_{Hb}^{{λ}_{1}} - {ɛ}_{Hb}^{{λ}_{2}}}{( {ɛ}_{HbO}^{{λ}_{2}} - {ɛ}_{Hb}^{{λ}_{2}} ) - \frac{{I}^{{λ}_{2}} ( r )}{{I}^{{λ}_{1}} ( r )} \cdot ( {ɛ}_{HbO}^{{λ}_{1}} - {ɛ}_{Hb}^{{λ}_{1}} )} \qquad formula ( 1 )$$

Here, I^{λ}₁(r) is a measured value based on the photoacoustic wave generated by the light irradiation of the first wavelength λ₁, and I^{λ}₂(r) is a measured value based on the photoacoustic wave generated by the light irradiation of the second wavelength λ₂. ε_{Hb}^{λ}₁ is a molar absorption coefficient [mm⁻¹mol⁻¹] of deoxyhemoglobin corresponding to the first wavelength λ₁, and ε_{Hb}^{k}₂ is a molar absorption coefficient [mm⁻¹mol⁻¹] of deoxyhemoglobin corresponding to the second wavelength λ₂. ε_{HbO}^{λ}₁ is a molar absorption coefficient [mm⁻¹mol⁻¹] of oxyhemoglobin corresponding to the first wavelength λ₁, and ε_{HbO}^{λ}₂ is a molar absorption coefficient [mm⁻¹mol⁻¹] of oxyhemoglobin corresponding to the second wavelength λ₂. r is a position. The measured values I^{λ}₁(r) and I^{λ}₂(r) may have absorption coefficients μₐ^{λ}₁(r) and μₐ^{λ}₂(r) or may be initial sound pressures P₀^{λ}₁(r) and P₀^{λ}₂(r).

Where the measured value based on the photoacoustic wave generated from the region where hemoglobin is present (blood vessel region) is substituted into the formula (1), the oxygen saturation degree (or an index correlated with the oxygen saturation degree) of hemoglobin is obtained as the calculated value Is(r). Meanwhile, where the measured value based on the acoustic wave generated from the region where the contrast agent is present (for example, a lymphatic vessel region) is substituted into the formula (1), the concentration distribution of a pseudo-contrast agent is obtained as the calculated value Is(r). Even when calculating the concentration distribution of the contrast agent, the numerical value of the molar absorption coefficient of hemoglobin may be used as it is in the formula (1). In the spectral image having the image value Is(r) obtained in this way, both the region where hemoglobin is present (blood vessel) and the region where the contrast agent is present (for example, a lymphatic vessel) inside the subject are visualized in a state of being separable (distinguishable) from each other.

In the present embodiment, the image value of the spectral image is calculated using the formula (1) for calculating the oxygen saturation degree, but when an index other than the oxygen saturation degree is calculated as the image value of the spectral image, a calculation method other than that using the formula (1) may be used. Since known indexes and methods for calculation thereof can be used, detailed explanation will be omitted.

Further, in the system according to the present invention, the spectral image may be an image showing the ratio of the first photoacoustic image based on the photoacoustic wave generated by the light irradiation of the first wavelength λ₁ and the second photoacoustic image based on the photoacoustic wave generated by the light irradiation of the second wavelength λ₂.

That is, the spectral images may be based on the ratio of the first photoacoustic image based on the photoacoustic wave generated by irradiation with light of the first wavelength λ₁ and the second photoacoustic image based on the photoacoustic wave generated by irradiation with light of the second wavelength λ₂. Since the image generated according to the modified formula (1) can also be expressed by the ratio of the first photoacoustic image and the second photoacoustic image, this generated image can be said to be an image (spectral image) based on the ratio of the first photoacoustic image and the second photoacoustic image.

In order to ascertain the three-dimensional structure of the contrast-enhanced object, the spectral image may represent an image representing a two-dimensional spatial distribution in the depth direction from the surface of the subject or may represent a three-dimensional spatial distribution.

### First Embodiment

The system configuration and the image processing method according to the present embodiment will be described hereinbelow.

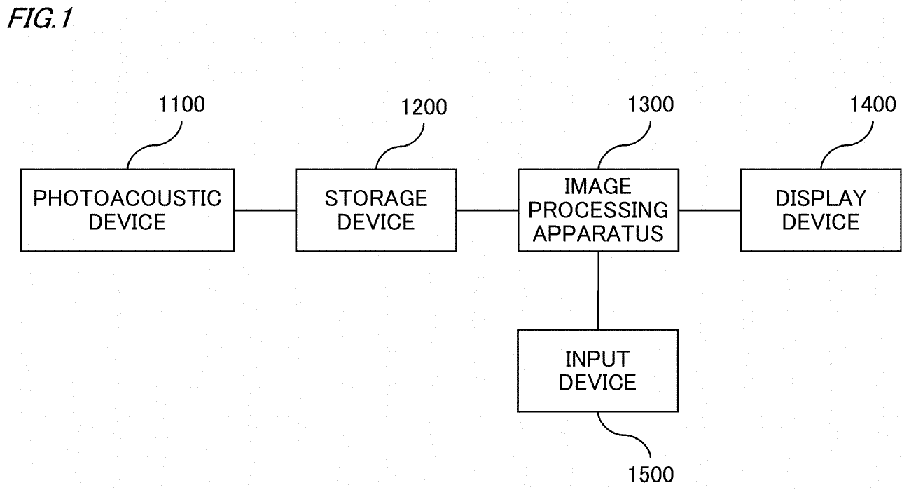

The system according to the present embodiment will be described with reference to FIG. 1. FIG. 1 is a block diagram showing a configuration of the system according to the present embodiment. The system according to the present embodiment includes a photoacoustic device 1100, a storage device 1200, an image processing apparatus 1300, a display device 1400, and an input device 1500. Data transfer between the devices may be performed by wire or wirelessly.

The photoacoustic device 1100 generates a photoacoustic image by capturing the image of a subject into which a contrast agent has been injected, and outputs the photoacoustic image to the storage device 1200. The photoacoustic device 1100 uses the received signal obtained by receiving the photoacoustic wave generated by the light irradiation to generate information on characteristic values corresponding to each of a plurality of positions in the subject. That is, the photoacoustic device 1100 generates a spatial distribution of characteristic value information derived from a photoacoustic wave as medical image data (photoacoustic image).

The storage device 1200 may be a storage medium such as a ROM (Read Only Memory), a magnetic disk, or a flash memory. Further, the storage device 1200 may be a storage server operating via a network such as PACS (Picture Archiving and Communication System).

The image processing apparatus 1300 processes the photoacoustic image stored in the storage device 1200, information incidental to the photoacoustic image, and the like. The image processing apparatus 1300 is a data acquisition means, an image acquisition means, and a display control means in the present invention.

A unit responsible for the computational function of the image processing apparatus 1300 can be configured of a processor such as a CPU and a GPU (Graphics Processing Unit), and a computational circuit such as an FPGA chip. These units may be configured not only of a single processor or computational circuit, but also may be configured of a plurality of processors or computational circuits.

A unit responsible for the storage function of the image processing apparatus 1300 can be configured of a non-temporary storage medium such as a ROM (Read Only Memory), a magnetic disk, or a flash memory. Further, a unit responsible for the storage function may be a volatile medium such as RAM (Random Access Memory). The storage medium in which the program is stored is a non-temporary storage medium. The unit having a storage function may be configured not only of one storage medium but may be configured of a plurality of storage media.

A unit responsible for the control function of the image processing apparatus 1300 is configured of a computational element such as a CPU. The unit responsible for the control function controls the operation of each configuration of the system. The unit responsible for the control function may control each configuration of the system by receiving instruction signals from various operations such as a start of measurement from the input unit. Further, the unit responsible for the control function may read a program code stored in a computer 150 to control the operation of each configuration of the system.

The display device 1400 is a liquid crystal display, an organic EL (Electro Luminescence) display, or the like. Further, the display device 1400 may display an image or a GUI for operating the device.

The input device 1500 is, for example, an operation console configured of a mouse, a keyboard, and the like that can be operated by the user. Further, the display device 1400 may be configured of a touch panel, and the display device 1400 may be used as the input device 1500.

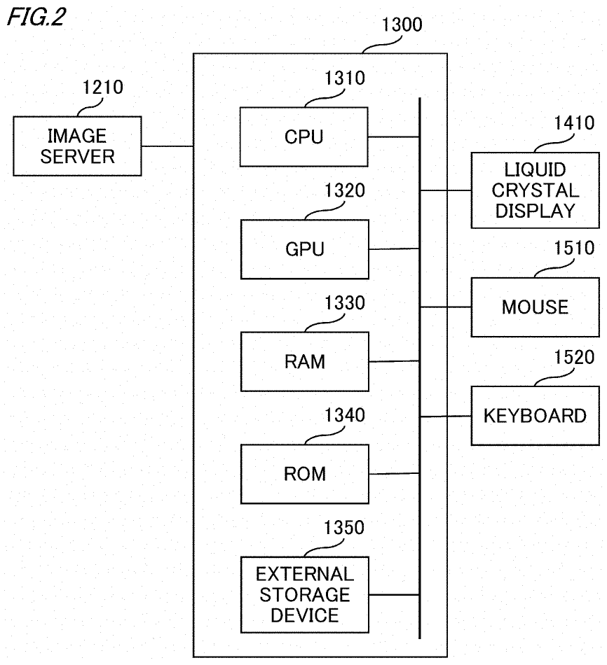

FIG. 2 shows a specific configuration example of the image processing apparatus 1300 according to the present embodiment. The image processing apparatus 1300 according to the present embodiment is configured of a CPU 1310, a GPU 1320, a RAM 1330, a ROM 1340, and an external storage device 1350. Further, a liquid crystal display 1410 as the display device 1400 and a mouse 1510 and a keyboard 1520 as an input device 1500 are connected to the image processing apparatus 1300. Further, the image processing apparatus 1300 is connected to an image server 1210 as a storage device 1200 such as a PACS (Picture Archiving and Communication System). As a result, the image data can be stored on the image server 1210, and the image data on the image server 1210 can be displayed on the liquid crystal display 1410.

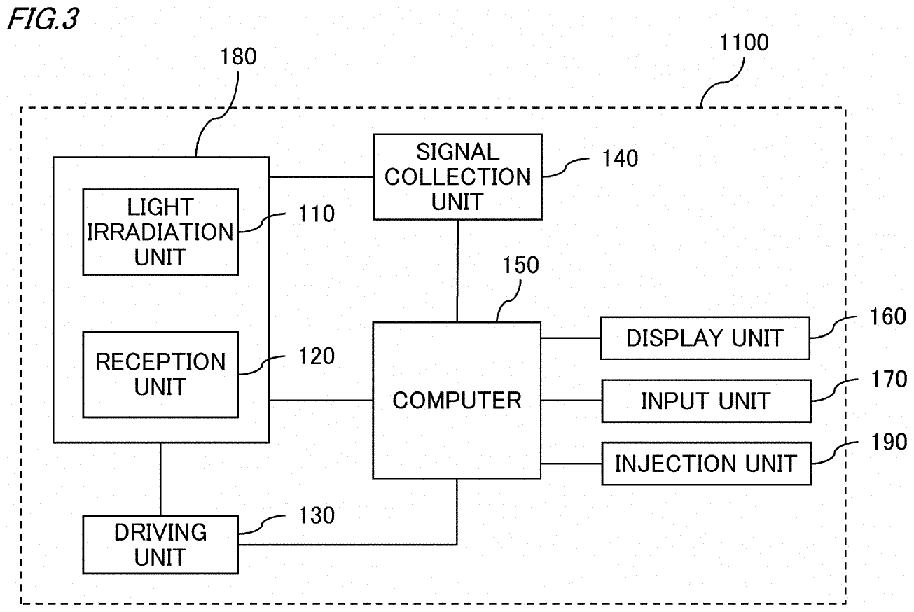

Next, a configuration example of devices included in the system according to the present embodiment will be described. FIG. 3 is a schematic block diagram of devices included in the system according to the present embodiment.

The photoacoustic device 1100 according to the present embodiment includes a driving unit 130, a signal collection unit 140, the computer 150, a probe 180, and an injection unit 190.

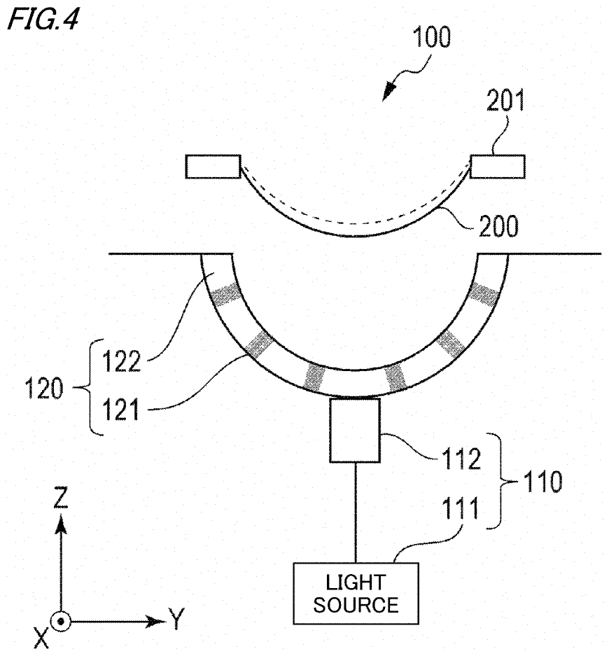

The probe 180 has a light irradiation unit 110 and a reception unit 120. FIG. 4 shows a schematic view of the probe 180 according to the present embodiment. The measurement target is a subject 100 into which the contrast agent has been injected by the injection unit 190.

The driving unit 130 drives the light irradiation unit 110 and the reception unit 120 to perform mechanical scanning. The light irradiation unit 110 irradiates the subject 100 with light, and an acoustic wave is generated in the subject 100. Acoustic waves generated by photoacoustic effects caused by light are also called photoacoustic waves.

The reception unit 120 outputs an electric signal (photoacoustic signal) as an analog signal by receiving the photoacoustic wave.

The signal collection unit 140 converts the analog signal outputted from the reception unit 120 into a digital signal and outputs the digital signal to the computer 150.

The computer 150 stores the digital signal outputted from the signal collection unit 140 as signal data derived from the photoacoustic wave. The computer 150 generates a photoacoustic image by performing signal processing on the stored digital signal. Further, the computer 150 outputs the photoacoustic image to the display unit 160 after performing image processing on the obtained photoacoustic image.

The display unit 160 displays an image based on the photoacoustic image. The display image is stored in the storage device 1200 such as a memory in the computer 150 or a data management system connected to a modality via a network, based on a storage instruction from the user or the computer 150.

The computer 150 also performs drive control of the configuration included in the photoacoustic device. Further, the display unit 160 may display a GUI or the like in addition to the image generated by the computer 150. The input unit 170 is configured so that the user can input information. The user can operate the start and end of measurement, the save instruction of the created image, and the like by using the input unit 170.

Hereinafter, details of each configuration of the photoacoustic device 1100 according to the present embodiment will be described.

<Light Irradiation Unit 110>

The light irradiation unit 110 includes a light source 111 that emits light and an optical system 112 that guides the light emitted from the light source 111 to the subject 100. The light includes pulsed light such as a so-called rectangular wave or triangular wave.

The pulse width of the light emitted by the light source 111 is preferably not more than 100 ns in consideration of the heat confinement condition and the stress containment condition. Further, the wavelength of light may be in the range of about 400 nm to 1600 nm. When imaging a blood vessel with high resolution, a wavelength (at least 400 nm, not more than 700 nm) that is highly absorbed by the blood vessel may be used. When imaging a deep part of a living body, light having a wavelength (at least 700 nm, not more than 1100 nm) that is typically less absorbed in the background tissue of the living body (water, fat, and the like) may be used.

The light source 111 is a laser, a light emitting diode, or the like. Further, when measuring using light having a plurality of wavelengths, a light source with a variable wavelength may be used. When irradiating a subject with a plurality of wavelengths, it is possible to prepare a plurality of light sources that generate light having different wavelengths and irradiate the subject alternately from the respective light sources. Even when a plurality of light sources is used, these light sources are collectively expressed as a light source. As the laser, various lasers such as a solid-state laser, a gas laser, a dye laser, and a semiconductor laser can be used. For example, a pulse laser such as an Nd:YAG laser or an alexandrite laser may be used as a light source. Further, a Ti:sa laser or an OPO (Optical Parametric Oscillators) laser using Nd:YAG laser light as excitation light may be used as a light source. Further, a flash lamp or a light emitting diode may be used as the light source 111. Further, a microwave source may be used as the light source 111.

Optical elements such as lenses, mirrors, and optical fibers can be used for the optical system 112. When a breast or the like is the subject 100, the light emitting unit of the optical system may be configured of a diffuser plate or the like that diffuses the light in order to irradiate the subject with a larger beam diameter of the pulsed light. Meanwhile, in a photoacoustic microscope, in order to increase the resolution, the light emitting unit of the optical system 112 may be configured of a lens or the like, and irradiation may be performed with a focused beam.

The subject 100 may be also directly irradiated with light from the light source 111, without providing the light irradiation unit 110 with the optical system 112.

<Reception Unit 120>

The reception unit 120 includes a transducer 121 that outputs an electric signal upon reception of an acoustic wave, and a support 122 that supports the transducer 121. Further, the transducer 121 may be a transmission means for transmitting an acoustic wave. The transducer as a receiving means and the transducer as a transmitting means may be a single (common) transducer or may be separate configurations.

As a member constituting the transducer 121, a piezoelectric ceramic material typified by PZT (lead zirconate titanate), a polymer piezoelectric membrane material typified by PVDF (polyvinylidene fluoride), or the like can be used. Further, an element other than the piezoelectric element may be used. For example, a transducer using a capacitance type transducer (CMUT: Capacitive Micro-machined Ultrasonic Transducers) can be used. Any transducer may be used as long as the transducer can output an electric signal by receiving an acoustic wave. Further, the signal obtained by the transducer is a time-resolved signal. That is, the amplitude of the signal obtained by the transducer represents a value based on the sound pressure received by the transducer at each time (for example, a value proportional to the sound pressure).

The frequency components constituting the photoacoustic wave are typically from 100 KHz to 100 MHz, and the transducer 121 capable of detecting these frequencies may be adopted.

The support 122 may be configured of a metal material having high mechanical strength or the like. In order to obtain a large amount of irradiation light incident on the subject, the surface of the support 122 on the subject 100 side may be mirror-finished or processed to ensure light scattering. In the present embodiment, the support 122 is configured to have a hemispherical shell shape and to be capable of supporting a plurality of transducers 121 on the hemispherical shell. In this case, the directivity axes of the transducers 121 arranged on the support 122 gather near the center of curvature of the hemisphere. Then, when an image is created using the signals outputted from the plurality of transducers 121, the image quality near the center of curvature becomes high. The support 122 may have any configuration as long as the transducer 121 can be supported. The support 122 may have a plurality of transducers arranged side by side in a plane or curved surface such as a 1D array, a 1.5D array, a 1.75D array, or a 2D array. The plurality of transducers 121 correspond to the plurality of receiving means.

Further, the support 122 may function as a container that stores an acoustic matching material. That is, the support 122 may be used as a container for arranging the acoustic matching material between the transducer 121 and the subject 100.

Further, the reception unit 120 may include an amplifier that amplifies a time-series analog signal outputted from the transducer 121. Further, the reception unit 120 may include an A/D converter that converts a time-series analog signal output from the transducer 121 into a time-series digital signal. That is, the reception unit 120 may include a signal collection unit 140 described hereinbelow.

The space between the reception unit 120 and the subject 100 is filled with a medium through which photoacoustic waves can propagate. This medium is a material capable of propagating acoustic waves, the material having matching acoustic characteristics at the interface with the subject 100 and the transducer 121 and having as high a transmittance of photoacoustic waves as possible. For example, this medium may be water, ultrasonic gel, or the like.

FIG. 4 shows a side view of the probe 180. The probe 180 according to the present embodiment has the reception unit 120 in which a plurality of transducers 121 is arranged three-dimensionally on the hemispherical support 122 having an opening. Further, a light emitting unit of the optical system 112 is arranged at the bottom of the support 122.

In the present embodiment, as shown in FIG. 4, the shape of the subject 100 is retained by contact with a holding portion 200.

The space between the reception unit 120 and the holding portion 200 is filled with a medium through which photoacoustic waves can propagate. This medium is a material capable of propagating photoacoustic waves, the material having matching acoustic characteristics at the interface with the subject 100 and the transducer 121 and having as high a transmittance of photoacoustic waves as possible. For example, this medium may be water, ultrasonic gel, or the like.

The holding portion 200 as a holding means holds the shape of the subject 100 during the measurement. By holding the subject 100 with the holding portion 200, it is possible to suppress the movement of the subject 100 and keep the position of the subject 100 in the holding portion 200. As the material of the holding portion 200, a resin material such as a polycarbonate, polyethylene, or polyethylene terephthalate can be used.

The holding portion 200 is attached to a mounting portion 201. The mounting portion 201 may be configured so that a plurality of types of holding portions 200 can be exchanged according to the size of the subject. For example, the mounting portion 201 may be configured to be replaceable according to holding portions that differ in a radius of curvature, a center of curvature, and the like.

<Driving Unit 130>

The driving unit 130 changes the relative position between the subject 100 and the reception unit 120. The driving unit 130 includes a motor such as a stepping motor that generates a driving force, a drive mechanism that transmits the driving force, and a position sensor that detects the position information of the reception unit 120. The drive mechanism includes a lead screw mechanism, a link mechanism, a gear mechanism, a hydraulic mechanism, and the like. Further, the position sensor is a potentiometer or the like using an encoder, a variable resistor, a linear scale, a magnetic sensor, an infrared sensor, an ultrasonic sensor, or the like.

The driving unit 130 is not limited to changing the relative position between the subject 100 and the reception unit 120 in the XY direction (two-dimensional) and may change the relative position one-dimensionally or three-dimensionally as well.

The driving unit 130 may fix the reception unit 120 and move the subject 100 as long as the relative position between the subject 100 and the reception unit 120 can be changed. When moving the subject 100, a configuration, etc. can be considered in which the subject 100 is moved by moving the holding portion that holds the subject 100. Further, both the subject 100 and the reception unit 120 may be moved.

The driving unit 130 may be configured to move the relative position continuously, or by step and repeat. The driving unit 130 may be an electric stage that is moved according to a programmed trajectory or a manual stage.

Further, in the present embodiment, the driving unit 130 simultaneously drives the light irradiation unit 110 and the reception unit 120 to perform scanning but may drive only the light irradiation unit 110 or only the reception unit 120.

When the probe 180 is of a handheld type provided with a grip portion, the photoacoustic device 1100 does not have to have the driving unit 130.

<Signal Collection Unit 140>

The signal collection unit 140 includes an amplifier that amplifies an electric signal that is an analog signal outputted from the transducer 121, and an A/D converter that converts an analog signal outputted from the amplifier into a digital signal. The digital signal outputted from the signal collection unit 140 is stored in the computer 150. The signal collection unit 140 is also called a Data Acquisition System (DAS). In the present description, an electric signal is a concept inclusive of both an analog signal and a digital signal. A photodetection sensor such as a photodiode may detect light emission from the light irradiation unit 110, and the signal collection unit 140 may start the above process in synchronization with the detection result as a trigger.

<Computer 150>

The computer 150 as an information processing device is configured of the same hardware as the image processing apparatus 1300. That is, a unit responsible for the computational function of the computer 150 is configured of a processor such as a CPU and a GPU (Graphics Processing Unit), and a computational circuit such as an FPGA (Field Programmable Gate Array) chip. These units may be configured not only of a single processor or computational circuit, but also may be configured of a plurality of processors or computational circuits.

A unit responsible for the storage function of the computer 150 may be a volatile medium such as RAM (Random Access Memory). The storage medium in which the program is stored is a non-temporary storage medium. The unit having the storage function of the computer 150 may be configured not only of one storage medium but may be configured of a plurality of storage media.

A unit responsible for the control function of the computer 150 is configured of a computational element such as a CPU. The unit responsible for the control function of the computer 150 controls the operation of each configuration of the photoacoustic device. The unit responsible for the control function of the computer 150 may control each configuration of the photoacoustic device by receiving instruction signals from various operations such as a start of measurement from the input unit 170. Further, the unit responsible for the control function of the computer 150 reads a program code stored in the unit responsible for the storage function to control the operation of each configuration of the photoacoustic device. That is, the computer 150 can function as a control device for the system according to the present embodiment.

The computer 150 and the image processing apparatus 1300 may be configured with the same hardware. One piece of hardware may be responsible for both the functions of the computer 150 and the image processing apparatus 1300. That is, the computer 150 may take on the function of the image processing apparatus 1300. Further, the image processing apparatus 1300 may take on the function of the computer 150 as an information processing device.

<Display Unit 160>

The display unit 160 is a liquid crystal display, an organic EL (Electro Luminescence) display, or the like. Further, the display unit 160 may display a GUI for operating an image or a device.

The display unit 160 and the display device 1400 may have the same display. That is, one display may have the functions of both the display unit 160 and the display device 1400.

<Input Unit 170>

The input unit 170 is, for example, an operation console composed of a mouse, a keyboard, and the like that can be operated by the user. Further, the display unit 160 may be configured by a touch panel, and the display unit 160 may be used as the input unit 170.

The input unit 170 and the input device 1500 may be the same device. That is, one device may have the functions of both the input unit 170 and the input device 1500.

<Introduction Unit 190>

The injection unit 190 is configured so that a contrast agent can be injected from the outside of the subject 100 into the subject 100. For example, the injection unit 190 can include a container for the contrast agent and an injection needle that pierces the subject. However, the injection unit 190 may have various configurations as long as the contrast agent can be injected into the subject 100. In this case, the injection unit 190 may be, for example, a known injection system or an injector. The computer 150 as a control device may inject the contrast agent into the subject 100 by controlling the operation of the injection unit 190. Further, the contrast agent may be injected into the subject 100 by the user operating the injection unit 190.

<Subject 100>

The subject 100 does not constitute a system but will be described hereinbelow. The system according to the present embodiment can be used for the purpose of diagnosing malignant tumors and vascular diseases of humans and animals, follow-up of chemotherapy, and the like. Therefore, the subject 100 is assumed to be a living body, specifically, a target site for diagnosis such as the breast of a human body or an animal, various organs, a vascular network, a head, a neck, an abdomen, and limbs including fingers or toes. For example, where a human body is the measurement target, the target of the light absorber is oxyhemoglobin or deoxyhemoglobin, a blood vessel containing a large amount thereof, or a new blood vessel formed in the vicinity of a tumor. The target of the light absorber may be plaque on the carotid artery wall, melanin, collagen, lipid, etc. contained in skin or the like. Further, the contrast agent injected into the subject 100 can be a light absorber. Contrast agents used for photoacoustic imaging include dyes such as indocyanine green (ICG) and methylene blue (MB), gold fine particles, and mixtures thereof, or substances introduced from the outside in which those are accumulated or chemically modified. Further, the subject 100 may be a phantom that imitates a living body.

Each configuration of the photoacoustic device may be configured as a separate device, or the configurations may be configured as an integrated device. Further, at least a part of the photoacoustic device may be configured as one integrated device.

Each device constituting the system according to the present embodiment may be configured with separate hardware, or all devices may be configured with one hardware. The functions of the system according to the present embodiment may be configured by any hardware.

Next, the image generation method according to the present embodiment will be described with reference to the flowchart shown in FIG. 5. The flowchart shown in FIG. 5 includes a step of showing the operation of the system according to the present embodiment and a step of showing the operation of a user such as a doctor.

First, in step S100, the computer 150 acquires examination order information transmitted from a system such as HIS (Hospital Information System) or RIS (Radiology Information System). The examination order information is information including information such as the type of modality to be used for the examination and the contrast agent to be used for the examination.

Next, in step S200, the computer 150 acquires information about the contrast agent. In this step, for example, the user of the photoacoustic device uses the input unit 170 to input the type of the contrast agent to be used for the examination and the concentration of the contrast agent. In this case, the computer 150 acquires information about the contrast agent via the input unit 170.

The computer 150 may read out information about the contrast agent from the examination order information acquired in step S100. The computer 150 may also acquire information about the contrast agent on the basis of at least one of the user's instruction and the examination order information.

Next, in step S300, the injection unit 190 injects the contrast agent into the subject. When the user injects the contrast agent into the subject using the injection unit 190, the user may notify the photoacoustic device 1100 of the injection of the contrast agent via the input unit 170. In this case, a signal indicating that the contrast agent has been injected may be transmitted from the input unit 170 to the computer 150. The injection unit 190 may also transmit a signal indicating that the contrast agent has been injected into the subject 100 to the computer 150. The contrast agent may be directly administered to the subject without using the injection unit 190. For example, the contrast agent may be administered by suction of the sprayed contrast agent by the living body as a subject.

Here, the concentration of ICG will be described with reference to a spectral image obtained by capturing, with a photoacoustic device, an image of a living body into which ICG has been injected.

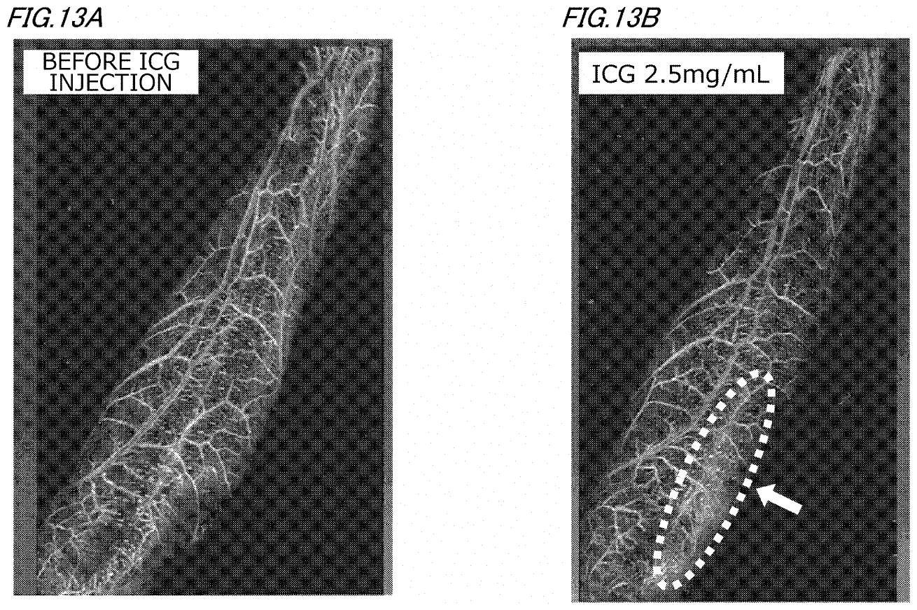

FIGS. 13 to 15 show spectral images obtained by capturing images when ICG was injected at different concentrations. In each of the captured images, 0.1 mL of ICG was injected subcutaneously or intradermally in a hand or feet. Since the ICG injected subcutaneously or intradermally is selectively taken up by the lymphatic vessels, the lumen of the lymphatic vessels is imaged. In each case, the images were captured within 5 min to 60 min after the injection of ICG. Further, each spectral image is generated from a photoacoustic image obtained by irradiating a living body with light having a wavelength of 797 nm and light having a wavelength of 835 nm.

FIG. 13A shows a spectral image of the extensor side of the right forearm when ICG was not injected. Meanwhile, FIG. 13B shows a spectral image of the extensor side of the right forearm when ICG having a concentration of 2.5 mg/mL was injected. Lymphatic vessels are visualized in the areas indicated by the dashed lines and arrows in FIG. 13B.

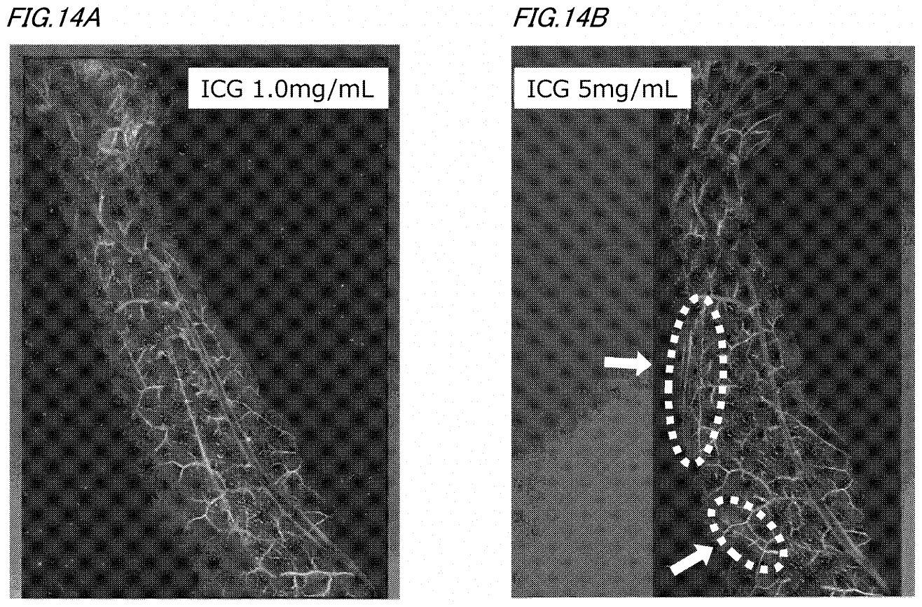

FIG. 14A shows a spectral image of the extensor side of the left forearm when ICG having a concentration of 1.0 mg/mL was injected. FIG. 14B shows a spectral image of the extensor side of the left forearm when ICG having a concentration of 5.0 mg/mL was injected. Lymphatic vessels are visualized in the areas indicated by the dashed lines and arrows in FIG. 14B.

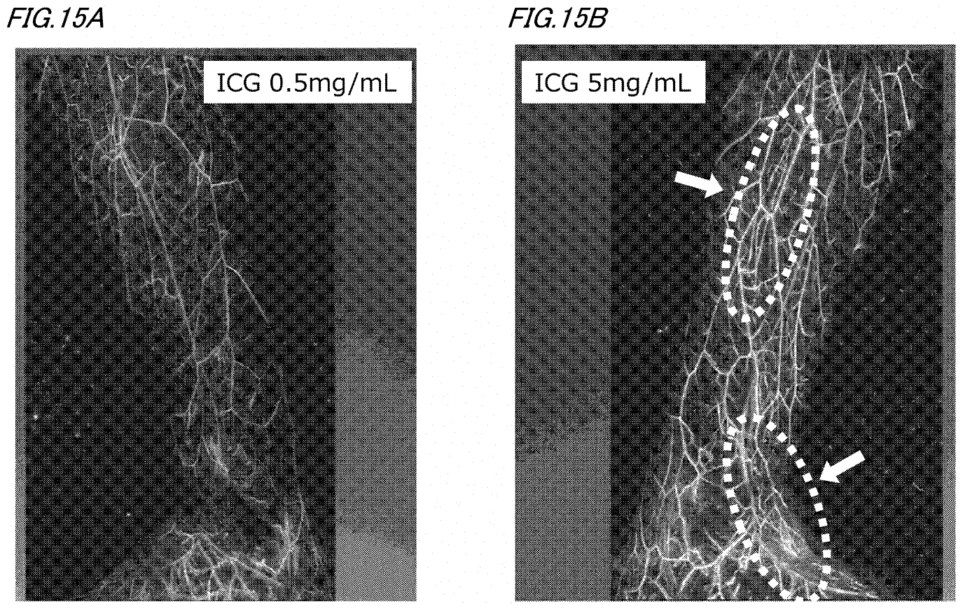

FIG. 15A shows a spectral image of the inside of the right lower leg when ICG having a concentration of 0.5 mg/mL was injected. FIG. 15B shows a spectral image of the inside of the left thigh when ICG having a concentration of 5.0 mg/mL was injected. Lymphatic vessels are visualized in the regions indicated by the dashed lines and arrows in FIG. 15B.

According to the spectral images shown in FIGS. 13 to 15, it is understood that increasing the concentration of ICG improves the visibility of the lymphatic vessels in the spectral image. Further, according to FIGS. 13 to 15, it is understood that lymphatic vessels can be satisfactorily visualized when the concentration of ICG is at least 2.5 mg/mL. That is, the lymphatic vessels on a line can be clearly visually recognized when the concentration of ICG is at least 2.5 mg/mL. Therefore, when ICG is used as the contrast agent, the concentration thereof may be at least 2.5 mg/mL. Considering the dilution of ICG in vivo, the concentration of ICG may be larger than 5.0 mg/mL. However, considering the solubility of diagonogreen, dissolution in an aqueous solution at a concentration of at least 10.0 mg/mL is difficult.

From the above, the concentration of ICG to be injected into the living body is preferably at least 2.5 mg/mL and not more than 10.0 mg/mL, and preferably at least 5.0 mg/mL and not more than 10.0 mg/mL.

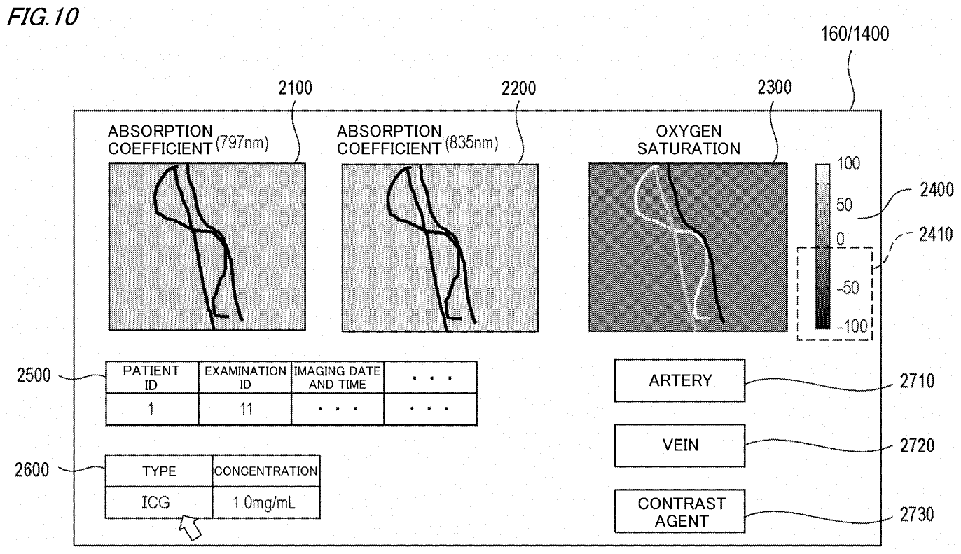

Therefore, the computer 150 may be configured to selectively receive an instruction from the user indicating the concentration of ICG in the above numerical range when ICG is inputted as the type of contrast agent in the GUI item 2600 shown in FIG. 10. That is, in this case, the computer 150 may be configured not to receive the user's instruction indicating the concentration of ICG outside the above numerical range. Therefore, the computer 150 may be configured not to receive the user's instruction indicating the concentration of ICG smaller than 2.5 mg/mL or greater than 10.0 mg/mL when information indicating that the type of contrast agent is ICG is acquired. The computer 150 may also be configured not to receive the user's instruction indicating the concentration of ICG smaller than 5.0 mg/mL or greater than 10.0 mg/mL when information indicating that the type of contrast agent is ICG is acquired.

In the computer 150, the GUI may be configured so that the user cannot instruct the concentration of ICG outside the above numerical range on the GUI. That is, the computer 150 may display the GUI so that the user cannot instruct the concentration of ICG outside the above numerical range on the GUI. For example, the computer 150 may display a pull-down on the GUI that can selectively indicate the concentration of ICG in the above numerical range. In the computer 150, the GUI may be configured to gray out and display the concentration of ICG other than the above numerical range in the pull-down, so that the grayed-out concentration cannot be selected.

Further, the computer 150 may notify an alert when the concentration of ICG outside the above numerical range is instructed by the user on the GUI. As the notification method, any method such as displaying an alert on the display unit 160, a sound, lighting or a lamp, or the like can be adopted.

Further, the computer 150 may display the above numerical range as the concentration of ICG to be injected into the subject on the display unit 160 when ICG is selected as the type of contrast agent on the GUI.

The concentration of the contrast agent to be injected into the subject is not limited to the numerical range shown herein, and a suitable concentration can be adopted according to the purpose. Further, although an example in which the type of contrast agent is ICG has been described herein, the above configuration can be similarly applied to other contrast agents.

By configuring the GUI in this way, it is possible to help the user to inject an appropriate contrast agent concentration into the subject according to the type of the contrast agent to be injected into the subject.

Next, in step S400, the wavelength of the irradiation light corresponding to the contrast agent is determined. The processing after this step may be performed after a while until the contrast agent is distributed to the contrast-enhanced object in the subject 100. In this step, the computer 150 determines the wavelength of the irradiation light on the basis of information about the contrast agent acquired in step S200. In the present embodiment, the computer 150 determines a plurality of wavelengths on the basis of information about the contrast agent in order to generate a spectral image.

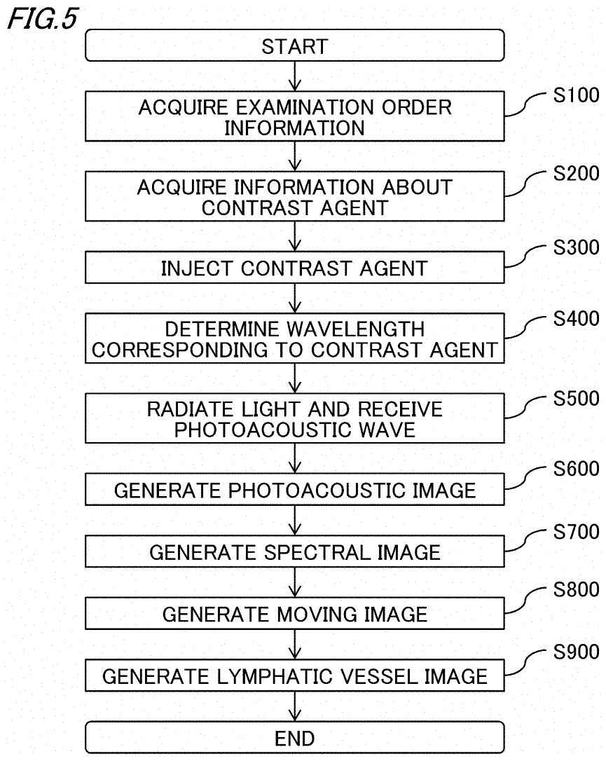

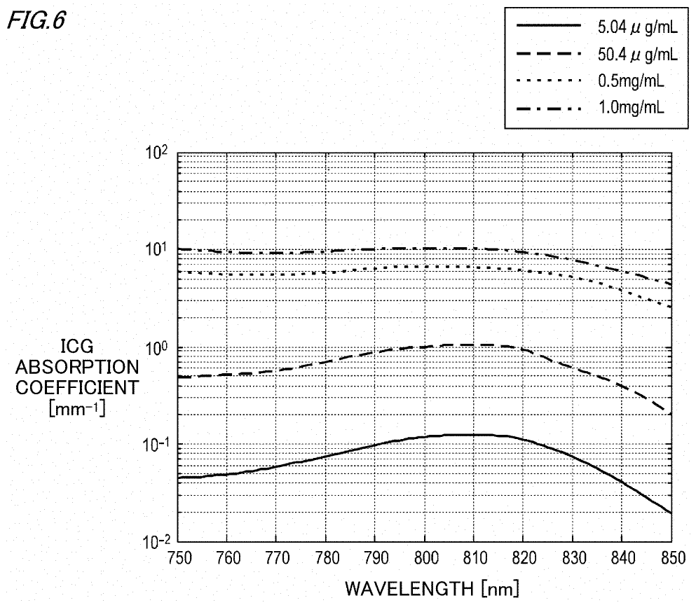

FIG. 6 is a spectrum diagram showing a change in the absorption coefficient spectrum when the concentration of ICG as a contrast agent is changed. The graph shown in FIG. 6 shows the spectra when the concentration of ICG is 5.04 μg/mL, 50.4 μg/mL, 0.5 mg/mL, and 1.0 mg/mL in order from the bottom. As shown in FIG. 6, it is understood that the degree of light absorption increases as the concentration of the contrast agent increases. Thus, since the ratio of the absorption coefficient at any two arbitrary wavelengths differs depending on the concentration of the contrast agent, it is necessary to determine an appropriate wavelength of the irradiation light according to the concentration of the contrast agent to be used.

The oxygen saturation degree in blood vessels (arteries and veins) in the living body is generally within the range of 60% to 100% in percentage display. Therefore, the wavelength (two wavelengths) of the light by which the subject is irradiated is preferably such that the oxygen saturation degree value (calculated value of the formula (1)) corresponding to the contrast agent in the spectral image becomes smaller than 60% or larger than 100%. By doing so, it becomes easy to distinguish between the image corresponding to the arteries and veins and the image corresponding to the contrast agent in the spectral image. For example, when ICG is used as a contrast agent, two wavelengths can be selected: a wavelength of at least 700 nm and smaller than 820 nm, and a wavelength of at least 820 nm and not more than 1020 nm. In this case, by generating a spectral image by the formula (1), the region of the contrast agent and the region of the blood vessel can be satisfactorily identified.

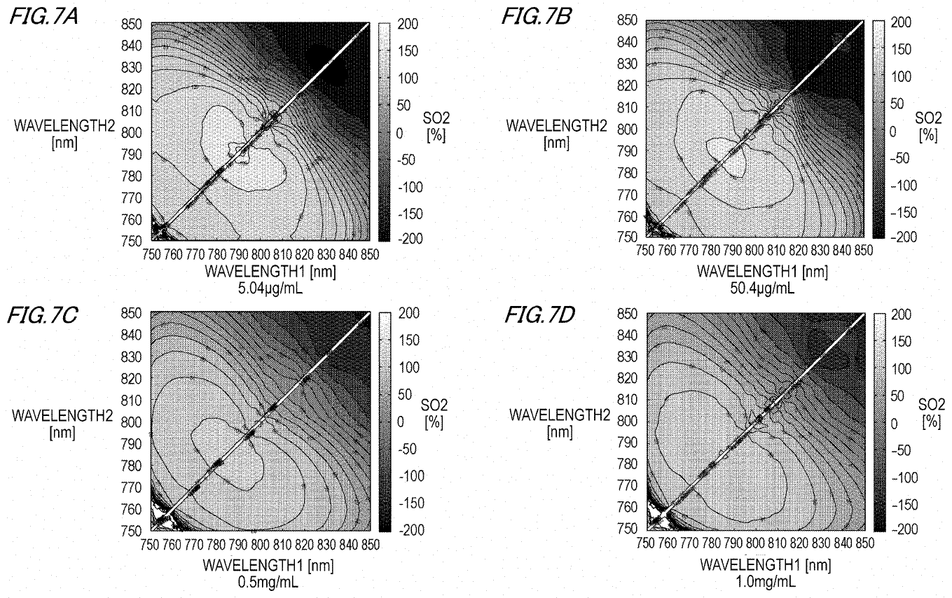

Next, a change in the image value corresponding to the contrast agent in the spectral image when a combination of wavelengths is changed will be described. FIG. 7 shows the simulation results of the image values (values calculated as pseudo oxygen saturation degree) corresponding to the contrast agent in the spectral image for each of the combinations of two wavelengths. The vertical and horizontal axes in FIG. 7 represent the first wavelength and the second wavelength, respectively. FIG. 7 shows contour lines of image values corresponding to the contrast agent in the spectral image.

FIGS. 7(a) to 7(d) show the image value corresponding to the contrast agent in the spectral images when the concentration of ICG is 5.04 μg/mL, 50.4 μg/mL, 0.5 mg/mL, and 1.0 mg/mL, respectively. As shown in FIG. 7, depending on the combination of wavelengths selected, the image value corresponding to the contrast agent in the spectral image may be 60% to 100%. When such a combination of wavelengths is used, it may be difficult to identify the blood vessel region and the contrast agent region in the spectral image. Therefore, among the wavelength combinations shown in FIG. 7, it is preferable to select a wavelength combination such that the image value corresponding to the contrast agent in the spectral image is smaller than 60% or larger than 100%. Furthermore, among the wavelength combinations shown in FIG. 7, it is preferable to select a wavelength combination such that the image value corresponding to the contrast agent in the spectral image becomes a negative value (minus). This is because the oxygen saturation degree in the blood cannot be a negative value, so that the region where the contrast agent is present can be easily identified.

Hereinafter, the region where the blood vessel is present is referred to as a blood vessel region, and the region where the contrast agent is present is referred to as a contrast agent region. The blood vessel region is the region corresponding to an artery or a vein, and the contrast agent region is the region corresponding to a lymphatic vessel.

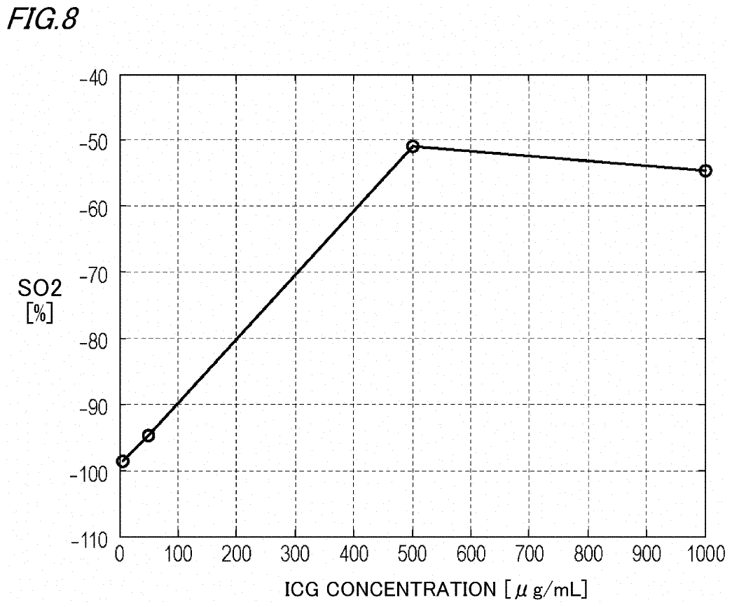

For example, a case can be considered where 797 nm is selected as the first wavelength and 835 nm is selected as the second wavelength. FIG. 8 is a graph showing the relationship between the concentration of ICG and the image value (value of the formula (1)) corresponding to the contrast agent in the spectral image when 797 nm is selected as the first wavelength and 835 nm is selected as the second wavelength. According to FIG. 8, when 797 nm is selected as the first wavelength and 835 nm is selected as the second wavelength, the image value corresponding to the contrast agent in the spectral image is negative at any concentration of 5.04 μg/mL to 1.0 mg/mL. Therefore, according to the spectral image generated by such a combination of wavelengths, since the oxygen saturation degree value in blood does not take a negative value in principle, the blood vessel region and the contrast agent region can be clearly identified.

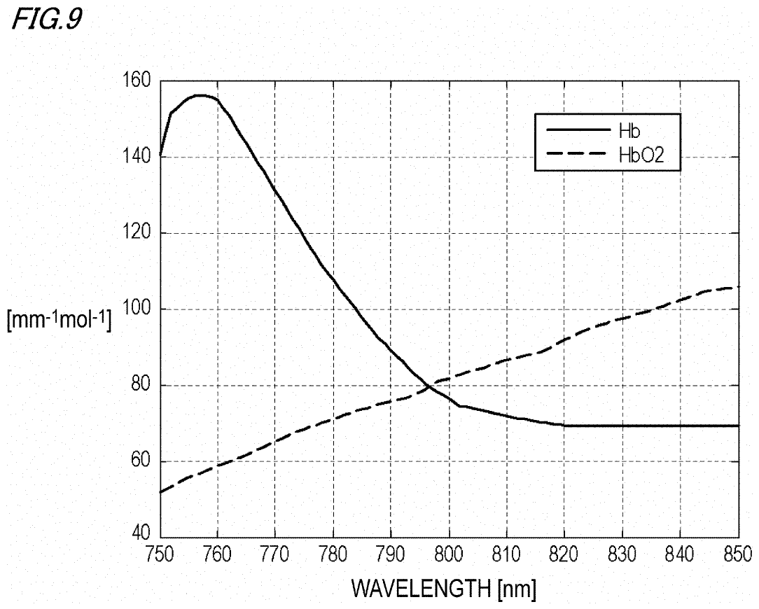

It has been explained that the wavelength of the irradiation light is determined based on the information about the contrast agent, but the absorption coefficient of hemoglobin may be also taken into consideration in determining the wavelength. FIG. 9 shows the spectra of the molar absorption coefficient of oxyhemoglobin (broken line) and the molar absorption coefficient of deoxyhemoglobin (solid line). In the wavelength range shown in FIG. 9, the magnitude relationship between the molar absorption coefficient of oxyhemoglobin and the molar absorption coefficient of deoxyhemoglobin is reversed at 797 nm as a boundary. That is, it is easy to ascertain a vein at a wavelength shorter than 797 nm, and it is easy to ascertain an artery at a wavelength longer than 797 nm. Lymphedema is treated by lymphaticovenous anastomosis (LVA) in which a bypass is created between lymphatic vessels and veins. For this preoperative examination, it is conceivable that photoacoustic imaging be performed for both the veins and the lymphatic vessels where the contrast agent has accumulated. In this case, the veins can be imaged more clearly by making at least one of the plurality of wavelengths smaller than 797 nm. Further, setting at least one of the plurality of wavelengths to a wavelength at which the molar absorption coefficient of deoxyhemoglobin is larger than the molar absorption coefficient of oxyhemoglobin is advantageous for imaging veins. Further, when a spectral image is generated from a photoacoustic image corresponding to two wavelengths, setting both of the two wavelengths to wavelengths at which the molar absorption coefficient of deoxyhemoglobin is larger than the molar absorption coefficient of oxyhemoglobin is also advantageous for imaging veins. The selection of these wavelengths enables accurate imaging of both the lymphatic vessels into which the contrast agent has been injected and the veins in the preoperative examination of lymphaticovenous anastomosis.

Where all of the plurality of wavelengths are such that the absorption coefficient of the contrast agent is higher than that of blood, the oxygen saturation degree accuracy of blood is lowered due to artifacts derived from the contrast agent. Therefore, in order to reduce the artifacts derived from the contrast agent, at least one of the plurality of wavelengths may be a wavelength at which the absorption coefficient of the contrast agent is smaller than the absorption coefficient of blood.

Here, the case of generating a spectral image according to the formula (1) has been described, but such an approach can be also adopted when generating a spectral image in which the image value corresponding to the contrast agent in the spectral image changes depending on the conditions of the contrast agent and the wavelength of the irradiation light.

The explanation is continued by returning to FIG. 5.

In step S500, the light irradiation unit 110 sets the wavelength determined in step S400 to the light source 111 and radiates light. The subject 100 is irradiated with the light generated from the light source 111 as pulsed light through the optical system 112. The pulsed light is absorbed inside the subject 100, and a photoacoustic wave is generated by the photoacoustic effect. At this time, the injected contrast agent also absorbs the pulsed light and generates a photoacoustic wave. The photoacoustic wave generated by the subject 100 is received by the transducer 121 and converted into an analog electric signal. The light irradiation unit 110 may transmit a synchronization signal to the signal collection unit 140 at the timing of irradiating with the pulsed light.

The light irradiation unit 110 similarly performs light irradiation for each of the plurality of wavelengths.

The user may input control parameters such as irradiation conditions of irradiation light (repetition frequency, wavelength, and the like), position of probe 180, and the like in advance by using the input unit 170. Further, the computer 150 may set the control parameters on the basis of user's instruction. The computer 150 may also move the probe 180 to a designated position by controlling the driving unit 130 on the basis of designated control parameters. Where image capturing at a plurality of positions is designated, the driving unit 130 first moves the probe 180 to the first designated position. The driving unit 130 may move the probe 180 to a pre-programmed position when the measurement start instruction is given.

The signal collection unit 140 starts the signal collecting operation upon receiving the synchronization signal transmitted from the light irradiation unit 110. That is, the signal collection unit 140 generates an amplified digital electric signal by amplifying and A/D converting the analog electric signal derived from the photoacoustic wave and outputted from the reception unit 120 and sends the generated digital signal to the computer 150. The computer 150 stores the signal transmitted from the signal collection unit 140. When image capturing at a plurality of scanning positions is designated, the step S500 is repeatedly executed at the designated scanning positions, and irradiation with the pulsed light and generation of a digital signal derived from an acoustic wave are repeated. The computer 150 may acquire the position information of the reception unit 120 at the time of light emission from the position sensor of the driving unit 130 and store the acquired position information by using the light emission as a trigger.

In the present embodiment, an example of irradiating with light of a plurality of wavelengths by time division has been described, but this light irradiation method is not limiting as long as signal data corresponding to each of the plurality of wavelengths can be acquired. For example, when encoding is performed by light irradiation, there may be a timing in which light of a plurality of wavelengths is radiated at substantially the same time.

Next, in step S600, the computer 150 generates a photoacoustic image on the basis of the stored signal data. The computer 150 outputs the generated photoacoustic image to the storage device 1200 for storage.

An analytical reconstruction methods such as a back projection method in a time domain and a back projection method in a Fourier domain and a model-based method (repetitive computation method) can be adopted as reconstruction algorithms for converting signal data into a two-dimensional or three-dimensional spatial distribution. For example, the back projection method in a time domain is Universal back-projection (UBP), Filtered back-projection (FBP), or Delay-and-Sum.

In the present embodiment, one three-dimensional photoacoustic image (volume data) is generated by image reconstruction using the photoacoustic signal obtained in one light irradiation of the subject. Further, by performing light irradiation a plurality of times and performing image reconstruction for each light irradiation, time-series three-dimensional image data (time-series volume data) are acquired. The three-dimensional image data obtained by image reconstruction for each light irradiation of a plurality of times of light irradiation are collectively referred to as three-dimensional image data corresponding to a plurality of times of light irradiation. Since the light irradiation is executed a plurality of times in a time series, the three-dimensional image data corresponding to the plurality of times of light irradiation constitute the time-series three-dimensional image data.

The computer 150 generates an image showing the initial sound pressure distribution (sound pressure generated at a plurality of positions) by performing reconstruction processing on the signal data. Further, the computer 150 may generate an image showing the absorption coefficient distribution by calculating the optical fluence distribution, by the light radiated to the subject 100, inside the subject 100 and dividing the initial sound pressure distribution by the optical fluence distribution. A known method can be adopted for calculating the optical fluence distribution.

The computer 150 can generate a photoacoustic image corresponding to each of a plurality of wavelengths of light. Specifically, the computer 150 can generate a first photoacoustic image corresponding to the first wavelength by performing reconstruction processing on the signal data obtained by light irradiation of the first wavelength. Further, the computer 150 can generate a second photoacoustic image corresponding to the second wavelength by performing reconstruction processing on the signal data obtained by light irradiation of the second wavelength. In this way, the computer 150 can generate a plurality of photoacoustic images corresponding to light of a plurality of wavelengths.

In the present embodiment, the computer 150 acquires the absorption coefficient distribution information corresponding to each of a plurality of light wavelengths as a photoacoustic image. Hereinafter, the absorption coefficient distribution information corresponding to the first wavelength will be referred to as a first photoacoustic image, and the absorption coefficient distribution information corresponding to the second wavelength will be referred to as a second photoacoustic image.

Although the system according to the present embodiment has been described as including the photoacoustic device 1100 that generates a photoacoustic image, the present invention can also be applied to a system that does not include the photoacoustic device 1100. The present invention can be applied to any system as long as the image processing apparatus 1300 can acquire a photoacoustic image. For example, the present invention can be applied to a system that does not include the photoacoustic device 1100 but includes the storage device 1200 and the image processing apparatus 1300. In this case, the image processing apparatus 1300 can acquire the photoacoustic image by reading out the designated photoacoustic image from a photoacoustic image group stored in advance in the storage device 1200.

Next, in step S700, the computer 150 generates a spectral image on the basis of a plurality of photoacoustic images corresponding to a plurality of wavelengths. The computer 150 outputs the spectral image to the storage device 1200 for storage in the storage device 1200. As described above, the computer 150 can generate an image showing information corresponding to the concentration of a substance constituting a subject, such as a contrast agent administered to the subject and the glucose concentration, collagen concentration, melanin concentration, and volume fraction of fat and water inherent to the subject, as a spectral image. Further, the computer 150 may generate an image representing the ratio of the first photoacoustic image corresponding to the first wavelength and the second photoacoustic image corresponding to the second wavelength as a spectral image. In the present embodiment, the computer 150 uses the first photoacoustic image and the second photoacoustic image to generate a spectral image showing the oxygen saturation degree according to the formula (1).

The image processing apparatus 1300 may acquire a spectral image by reading a designated spectral image from a group of spectral images stored in advance in the storage device 1200. The image processing apparatus 1300 may also acquire a spectral image by reading at least one of a plurality of photoacoustic images, which has been used for generating the read spectral image, from a group of photoacoustic images stored in advance in the storage device 1200.

Here, the problems arising when imaging lymphatic vessels in a living body by spectral images will be described.

Since the region where the contrast agent injected into the body is present can be visualized by a spectral image, the lymphatic vessel into which the contrast agent has been injected can be visualized. However, the position of the lymphatic vessels may not be shown correctly with only one image. This is because the flow of lymphatic fluid is not as constant as blood.

Blood is constantly circulated by the beating of the heart, but the lymphatic vessels do not have a common organ that acts as a pump, and the lymphatic fluid is transported by contraction of smooth muscles present in the lymphatic vessel walls that constitute a lymphatic vessel. In addition to the contraction of the smooth muscles of the lymphatic vessel wall that occurs once every few tens of seconds to several minutes, the lymphatic fluid moves due muscle contraction that occurs with human movement, pressure caused by relaxation, a pressure change caused by breathing, and external massage stimulation. Therefore, the movement timing of the lymphatic fluid is not constant, and the lymphatic fluid flows intermittently at irregular intervals such as once every several tens of seconds to several minutes. Even if a spectral image is acquired when the lymphatic fluid is not moving, there is a concern that because a sufficient amount of contrast agent is not present in the lymphatic vessel, the lymphatic vessel cannot be visualized, or only a part of the lymphatic vessel can be visualized.

Therefore, in the system according to the present embodiment, a plurality of spectral images (a plurality of first image data) along the time series is acquired in a predetermined period, and a region in which a lymphatic vessel is present (that is, the region through which the contrast agent passes) is extracted based on the acquired plurality of spectral images. In the present embodiment, the photoacoustic device 1100 acquires a plurality of spectral images along the time series in the processes of steps S500 to S700 and stores the acquired spectral images in the storage device 1200. The predetermined period is preferably longer than the cycle in which the movement of lymphatic fluid occurs (for example, longer than about 40 sec to 2 min).

Step S800 is a step of generating a moving image on the basis of the plurality of spectral images.

By displaying the plurality of spectral images as moving images, the user of the device can observe how the lymphatic fluid moves. However, since the lymphatic fluid flows intermittently in the lymphatic vessels, only some spectral images among the plurality of spectral images acquired in time series can be used to confirm the flow of the lymphatic fluid. That is, when the spectral image is displayed by the moving image, the user must keep looking at the screen until the movement of the lymphatic fluid occurs. Further, since the movement cycle of the lymphatic fluid (contrast agent) takes a short time, it is difficult for the user to accurately ascertain the position of the lymphatic vessel on the screen.

Accordingly, in the present embodiment, after executing step S800, in step S900, the image processing apparatus 1300 generates a still image (second image data) showing the position of the lymphatic vessel on the basis of the plurality of spectral images.

First, the process of step S800 will be described.

In step S800, the image processing apparatus 1300 acquires the plurality of spectral images stored in the storage device 1200 and generates a moving image.

Specifically, image processing is performed on each frame of the spectral image so that the contrast agent region and other regions can be identified based on the information about the contrast agent acquired in advance, and the processed image is outputted to the display device 1400. As the rendering method, any method such as maximum value projection method (MIP: Maximum Integrity Projection), volume rendering, and surface rendering can be adopted. Here, setting conditions such as a display area and a line-of-sight direction when rendering a three-dimensional image in two dimensions can be arbitrarily designated according to the observation target.

Here, an example in which 797 nm and 835 nm are set as the wavelengths of the irradiation light and a spectral image is generated according to the formula (1) in step S700 will be considered. As shown in FIG. 8, when these two wavelengths are selected, the image value corresponding to the contrast agent in the generated spectral image becomes a negative value regardless of the concentration of ICG.

FIG. 10 shows an example of a GUI including an absorption coefficient image (first photoacoustic image) 2100 corresponding to a wavelength of 797 nm, an absorption coefficient image (second photoacoustic image) 2200 corresponding to a wavelength of 835 nm, and an oxygen saturation degree image (spectral image) 2300. The GUI is generated by the image processing apparatus 1300. In this example, both the photoacoustic image and the spectral image are displayed, but only the spectral image may be displayed. Further, the image processing apparatus 1300 may switch between the display of the photoacoustic image and the display of the spectral image based on the instruction of the user.

Reference numeral 2500 represents examination order information, and reference numeral 2600 represents information related to the contrast agent. Information acquired via an external device such as HIS or RIS or the input unit 170 is displayed on the interface.

As shown in FIG. 10, the image processing apparatus 1300 includes a color bar 2400 in the GUI as a color scale showing the relationship between the image value of the spectral image and the display color. The image processing apparatus 1300 may determine a numerical range of image values to be assigned to the color scale on the basis of information about the contrast agent (for example, information indicating that the type of contrast agent is ICG) and information indicating the wavelength of the irradiation light. For example, the image processing apparatus 1300 may determine a numerical range including the oxygen saturation degree of the artery, the oxygen saturation degree of the vein, and the image value (negative image value) corresponding to the contrast agent obtained with the formula (1). The image processing apparatus 1300 may determine a numerical range of −100% to 100% and set the color bar 2400 in which −100% to 100% are assigned to a color gradation that changes from blue to red. By such a display method, in addition to the arteriovenous identification, the contrast agent region (where the image value becomes a negative value) can also be identified. Further, the image processing apparatus 1300 may also display an indicator 2410 indicating the numerical range of the image value corresponding to the contrast agent on the basis of information about the contrast agent and information indicating the wavelength of the irradiation light. Here, in the color bar 2400, a negative value region is indicated by the indicator 2410 as a numerical range of image values corresponding to ICG. By displaying the color scale so that the display color corresponding to the contrast agent can be identified in this way, the contrast agent region in the spectral image can be easily identified.

At least one of hue, lightness, and chroma may be assigned to the image value of the spectral image, and the remaining parameters of hue, lightness, and chroma may be assigned to the image value of the photoacoustic image. For example, an image in which hue and chroma are assigned to the image value of the spectral image and lightness is assigned to the image value of the photoacoustic image may be displayed. At this time, where the lightness is assigned to the image value of the photoacoustic image when the image value of the photoacoustic image corresponding to the contrast agent is larger or smaller than the image value of the photoacoustic image corresponding to the blood vessel, it may be difficult to see both the blood vessel and the contrast agent. Therefore, a conversion table for conversion from the image value of the photoacoustic image to the lightness may be changed depending on the image value of the spectral image. For example, when the image value of the spectral image is included in the numerical range of the image value corresponding to the contrast agent, the lightness corresponding to the image value of the photoacoustic image may be made smaller than that corresponding to the blood vessel. That is, when the contrast agent region and the blood vessel region are compared, where the image value of the photoacoustic image is the same, the lightness of the contrast agent region may be made smaller than that of the blood vessel region. The conversion table is a table showing the lightness corresponding to each of a plurality of image values. Further, when the image value of the spectral image is included in the numerical range of the image value corresponding to the contrast agent, the lightness corresponding to the image value of the photoacoustic image may be larger than that corresponding to the blood vessel. That is, when the contrast agent region and the blood vessel region are compared, where the image value of the photoacoustic image is the same, the lightness of the contrast agent region may be larger than that of the blood vessel region. Further, the numerical range of the image value of the photoacoustic image that does not convert the image value of the photoacoustic image into lightness may differ depending on the image value of the spectral image.

The conversion table may be changed to one suitable for the type and concentration of the contrast agent and the wavelength of the irradiation light. Therefore, the image processing apparatus 1300 may determine a conversion table for conversion from the image value of the photoacoustic image to the lightness on the basis of the information about the contrast agent and the information indicating the wavelength of the irradiation light. When the image value of the photoacoustic image corresponding to the contrast agent is estimated to be larger than that corresponding to the blood vessel, the image processing apparatus 1300 may make the lightness corresponding to the image value of the photoacoustic image corresponding to the contrast agent to be smaller than that corresponding to the blood vessel. By contrast, when the image value of the photoacoustic image corresponding to the contrast agent is estimated to be smaller than that corresponding to the blood vessel, the image processing apparatus 1300 may make the lightness corresponding to the image value of the photoacoustic image corresponding to the contrast agent to be larger than that corresponding to the blood vessel.

The image processing apparatus 1300 displays the spectral image (reference numeral 2300) included in the GUI shown in FIG. 10 by a moving image. That is, a plurality of spectral images in a predetermined period is outputted as a continuous image.

The plurality of spectral images may be reproduced at the same frame rate as at the time of image capturing or may be reproduced at a different frame rate (for example, fast forward). Therefore, a window for the user to manually input the frame rate, a slide bar for the user to change the frame rate, and the like may be added to the GUI of FIG. 10. In general, the flow of lymphatic fluid is intermittent, with a cycle of tens of seconds to minutes. However, by making the frame rate of the moving image displayed on the display unit 160 variable, it is possible to fast-forward the displayed moving image so that the user can check the state of the fluid in the lymphatic vessel in a short time.

Further, it may be possible to repeatedly display a moving image within a predetermined time range. At that time, it is also preferable to add a GUI such as a window or a slide bar to FIG. 10 so that the user could designate the range which is to be repeatedly displayed. As a result, for example, the user can repeatedly observe the moving image by designating the period during which the contrast agent flows in the moving image data, and it becomes easier for the user to ascertain how the fluid flows in the lymphatic vessel.

Next, in step S900, the image processing apparatus 1300 acquires a plurality of spectral images (spectral images of a plurality of frames) stored in the storage device 1200, and generates an image representing a region in which a lymphatic vessel is present.

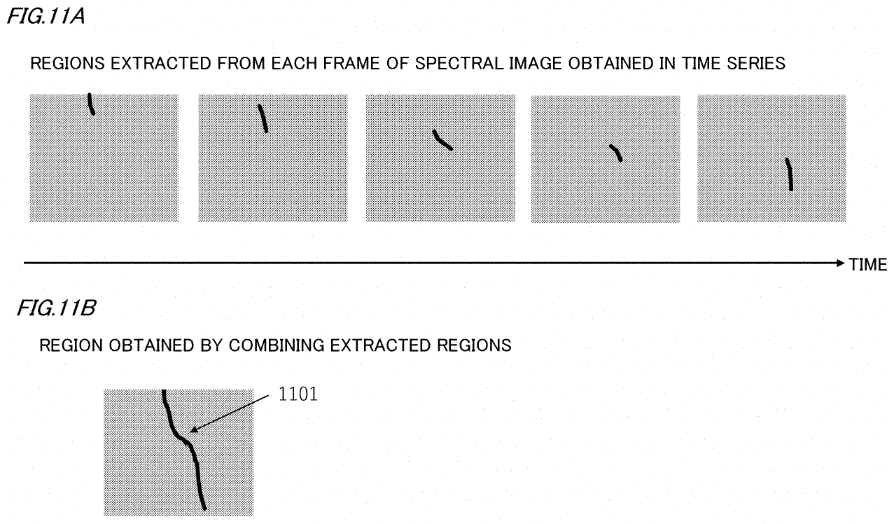

In this step, first, a region in which the image value is within a predetermined range is extracted for each of a plurality of spectral images obtained in time series. In the above-described example, a set of pixels for which the image value, which is the calculated value of the formula (1), is a negative value is extracted. As a result, as shown in FIG. 11A, a region (shown by a black line) is extracted for each frame of the moving image, that is, for each spectral image constituting the moving image. The extracted region is the region where the contrast agent is present in each frame. Although a two-dimensional image is illustrated in FIG. 11, when the spectral image is a three-dimensional spectral image, a region may be extracted from the three-dimensional space.

Then, the regions obtained for each frame are superimposed (combined) to generate a region corresponding to the lymphatic vessel. By superimposing the regions shown in FIG. 11A, the region corresponding to the lymphatic vessel (reference numeral 1101) as shown in FIG. 11B is obtained.

The image processing apparatus 1300 generates and outputs an image (second image data) representing the position of the lymphatic vessel on the basis of the region generated in this way. When generating an image showing the position of a lymphatic vessel, a hue corresponding to the original image value (that is, the image value of the spectral image) may be given, or highlighted display may be performed by applying marking. Further, the brightness corresponding to the absorption coefficient may be given. The absorption coefficient can be acquired from the photoacoustic image used to generate the spectral image.

The generated image may be outputted to the same screen as the GUI shown in FIG. 10 or may be outputted to another screen. The second image may be a three-dimensional image or a two-dimensional image. Further, an interface for storing the second image data generated as described above in the image server 1210, the storage device 1200, or the like may be added to the GUI shown in FIG. 10. Since the amount of the second image data is smaller than that of the first image data which are a moving image, the position of the lymphatic vessel can be easily ascertained even when a terminal having relatively low processing capacity is used.

According to the first embodiment, it becomes possible to provide a still image showing the position of a lymphatic vessel to a user such as a doctor. Since the lymphatic fluid (contrast agent) moves periodically, the position of a lymphatic vessel cannot be accurately presented when a plurality of spectral images is simply added (or averaged). Meanwhile, in the present embodiment, since the region in which the image value is in a predetermined range is extracted from each frame of the spectral image and combined, the information in the time direction is compressed. This makes it possible to accurately visualize the position of the lymphatic vessel.

In the illustrated embodiment, a region in which the image value of the spectral image is within a predetermined range is extracted, but region extraction may be performed in combination with other conditions. For example, a photoacoustic image (an image representing an absorption coefficient) corresponding to a spectral image may be referred to, and a region where the brightness value thereof is below a predetermined threshold value may be excluded. This is because even if the image value of the spectral image is within a predetermined range, the region where the absorption coefficient is small is likely to be noise. Further, the threshold value of the brightness value for performing filtering may be changed by the user.

In the present embodiment, a blood vessel and a contrast agent could be identified by selecting a wavelength such that the image value corresponding to the contrast agent (the value obtained with the formula (1)) becomes a negative value, but such image value is not limiting, and the image value corresponding to the contrast agent may be any value as long as the image value corresponding to the contrast agent makes it possible to identify the blood vessel and the contrast agent. For example, the image processing described in this step can be applied even when the image value of the spectral image (oxygen saturation degree image) corresponding to the contrast agent is smaller than 60% or larger than 100%.

Further, in the present embodiment, the wavelengths of the irradiation light (two wavelengths) were selected so that the image value of the pixel corresponding to the blood vessel region becomes positive and the image value of the pixel corresponding to the contrast agent region becomes negative, but any two wavelengths may be also selected so that the signs of both image values in the spectral image are reversed.

Further, the image processing apparatus 1300 may determine the contrast agent region in the spectral image on the basis of information related to the contrast agent and the information indicating the wavelengths of the irradiation light. For example, the image processing apparatus 1300 may determine a region having a negative image value in the spectral image as a contrast agent region. Then, the image processing apparatus 1300 may display the spectral image on the display device 1400 so that the contrast agent region and other regions can be identified. The image processing apparatus 1300 can employ identification display such as display of different colors for the contrast agent region and other regions, blinking on the contrast agent region, and displaying an indicator (for example, a frame) indicating the contrast agent region.

Switching to a display mode in which the image value corresponding to the ICG is selectively displayed may be performed by indicating an item 2730 corresponding to the display of the ICG displayed on the GUI shown in FIG. 10. For example, when the user selects the item 2730 corresponding to the display of the ICG, the image processing apparatus 1300 may selectively display the ICG region by selecting a voxel for which the image value is negative from the spectral image and selectively rendering the selected voxel. Similarly, the user may select an item 2710 corresponding to the display of an artery and an item 2720 corresponding to the display of a vein. Based on the user's instructions, the image processing apparatus 1300 may switch to a display mode in which an image value corresponding to an artery (for example, at least 90% and not more than 100%) or an image value corresponding to a vein (for example, at least 60% and less than 90%) is selectively displayed. The numerical range of the image value corresponding to an artery and the image value corresponding to a vein may be changed based on the user's instruction.

### Second Embodiment

In the first embodiment, in step S900, the extraction processing of each region was performed with respect to each frame of the spectral image acquired in time series, and a plurality of extracted regions was combined. Meanwhile, in the second embodiment, a plurality of frames of spectral images acquired in time series is referred to and a region satisfying the conditions is directly extracted within a predetermined period.

In the second embodiment, in step S900, a plurality of spectral images included in a predetermined period is selected, and a region (in the above-mentioned example, the region where the image value is negative) in which the image value falls within a predetermined range within the predetermined period is extracted. It can be said that the region in which the image value falls within the predetermined range within the predetermined period is thus the region through which the contrast agent has passed. The predetermined period is preferably longer than the cycle in which the movement of lymphatic fluid occurs (for example, longer than about 40 sec to 2 min).

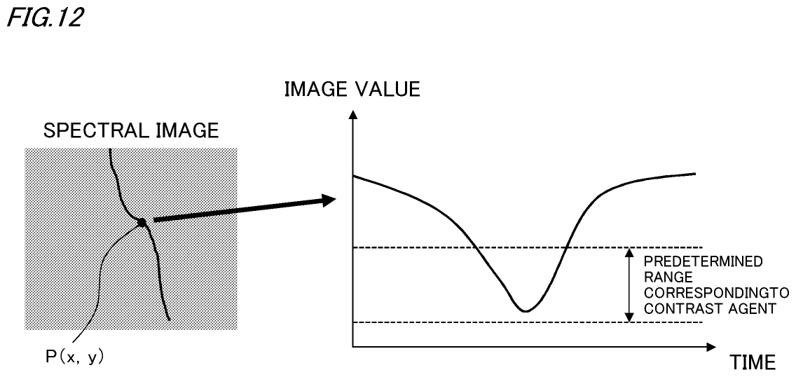

FIG. 12 is a diagram illustrating a change of an image value with time at a certain pixel P(x, y) in a spectral image within a predetermined period. The illustrated pixel is the extraction target because the image value is within a predetermined range.

As described above, the contrast agent region may be extracted based on the image value that changes within the predetermined period. When making the determination, a peak hold of the image value of the photoacoustic image may be performed within the predetermined period.

As a noise countermeasure, in the second embodiment as well, the region where the absorption coefficient is lower than the predetermined value may be excluded in the same manner as in the first embodiment. That is, a region in which the image value of the spectral image is within the predetermined range and the brightness of the corresponding photoacoustic image exceeds the threshold value may be the extraction target.

Further, as a noise countermeasure, a region where a state in which the above-mentioned conditions are satisfied is maintained for a certain period of time may be the extraction target. Further, the certain period of time may be adjustable by the user.

### Third Embodiment

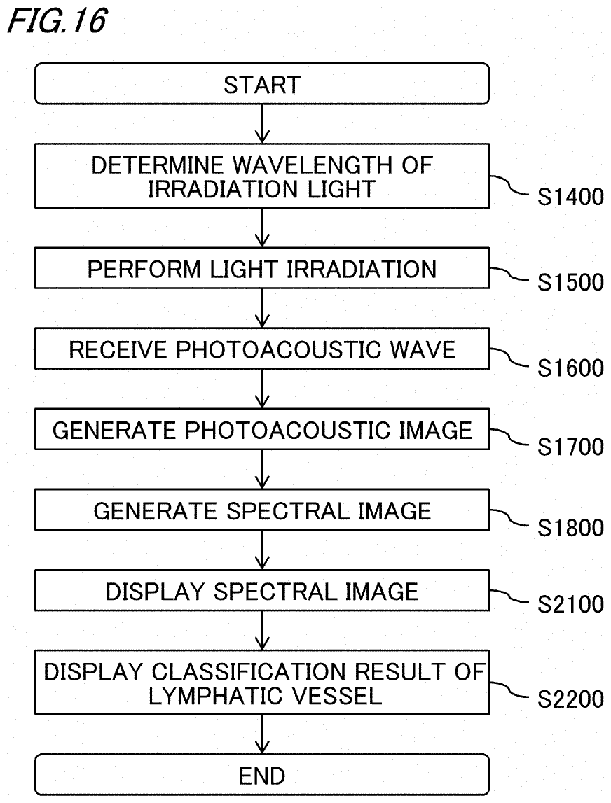

In the present embodiment, the image processing apparatus 1300 automatically classifies lymphatic vessels and estimates the state of the lymphatic vessels by analyzing the image data generated based on the received signal data of the photoacoustic wave generated from the inside of the subject by irradiating the subject with light. The image processing apparatus 1300 causes the display device 1400 to display the classification result. First, the image generation method according to the present embodiment will be described with reference to the flowchart shown in FIG. 16.

(S1400: Step of Determining Wavelength of Irradiation Light)

The computer 150 as a wavelength determining means determines the wavelength of the irradiation light on the basis of information about the contrast agent. In the present embodiment, a combination of wavelengths is determined so that the region corresponding to the contrast agent in the spectral image can be easily identified. The computer 150 can acquire information about the contrast agent which is inputted by a user such as a doctor using the input unit 170. Further, the computer 150 may store information about a plurality of contrast agents in advance and acquire information about the contrast agent that has been set by default from the stored information.

FIG. 10 shows an example of the GUI displayed on the display unit 160 in the third embodiment. In an item 2500 of the GUI, examination order information such as a patient ID, an examination ID, and an imaging date and time is displayed. The item 2500 may be provided with a display function for displaying examination order information acquired from an external device such as HIS or RIS, or an input function for allowing the user to input examination order information using the input unit 170. Information about the contrast agent such as the type of the contrast agent and the concentration of the contrast agent is displayed on the item 2600 of the GUI. The item 2600 may be provided with a display function for displaying information about the contrast agent acquired from an external device such as HIS or RIS, and an input function for allowing the user to input information about the contrast agent using the input unit 170. In item 2600, information about the contrast agent such as the type and concentration of the contrast agent may be inputted by a method such as pull-down from a plurality of options. The GUI shown in FIG. 10 may be displayed on the display device 1400.

When the image processing apparatus 1300 does not receive an input instruction of the information related to the contrast agent from the user, the information related to the contrast agent that has been set by default may be acquired from the information related to the plurality of contrast agents. In the present embodiment, the case where ICG is set as the type of the contrast agent and 1.0 mg/mL is set as the concentration of the contrast agent by default will be described. In the present embodiment, the type and concentration of the contrast agent set by default are displayed in the item 2600 of the GUI, but the information related to the contrast agent may not be set by default. In this case, the information related to the contrast agent may not be displayed in the item 2600 of the GUI on the initial screen.

A change in the image value corresponding to the contrast agent in the spectral image when the combination of wavelengths is changed is the same as in the explanation illustrated by FIGS. 7 and 8, and the explanation thereof will be omitted. Further, the wavelength may be determined in consideration of the absorption coefficient of hemoglobin as described with reference to FIG. 9.

(S1500: Step of Irradiation with Light)

The light irradiation unit 110 sets the wavelength determined in S1400 in the light source 111. The light source 111 emits light having a wavelength determined by S1400. Since the irradiation with light is the same as that of S500 in FIG. 5, detailed description thereof will be omitted.

(S1600: Step of Receiving Photoacoustic Waves)

When the signal collection unit 140 receives a synchronization signal transmitted from the light irradiation unit 110, the signal collection unit 140 starts the signal collecting operation. That is, the signal collection unit 140 generates an amplified digital electric signal by amplifying and AD converting the analog electric signal derived from the photoacoustic wave and outputted from the reception unit 120, and outputs the amplified digital electric signal to the computer 150. The computer 150 stores the signal transmitted from the signal collection unit 140. When image capturing at a plurality of scanning positions is designated, the steps S1500 and S1600 are repeatedly executed at the designated scanning positions, and the irradiation with pulsed light and the generation of digital signals derived from acoustic waves are repeated. Using the light emission as a trigger, the computer 150 may acquire and store the position information of the reception unit 120 at the time of light emission on the basis of the output from the position sensor of the driving unit 130.

In the present embodiment, an example of irradiation with light of a plurality of wavelengths in a time-division manner has been described, but this light irradiation method is not limiting as long as signal data corresponding to each of the plurality of wavelengths can be acquired. For example, when encoding is performed by light irradiation, there may be a timing in which irradiation with light of a plurality of wavelengths is performed at substantially the same time.

(S1700: Step of Generating Photoacoustic Image)

The computer 150 as a photoacoustic image acquisition means generates a photoacoustic image on the basis of stored signal data. The computer 150 outputs the generated photoacoustic image to the storage device 1200 for storage. In the present embodiment, one volume datum is generated by image reconstruction using the photoacoustic signal obtained by one light irradiation of the subject. Further, by performing light irradiation a plurality of times and reconstructing an image for each light irradiation, time-series three-dimensional volume data are acquired.

(S1800: Step of Generating Spectroscopic Image)

The computer 150 as a spectral image acquisition means generates a spectral image on the basis of a plurality of photoacoustic images corresponding to a plurality of wavelengths. Since the generation of the spectral image is the same as the process of step S700 in FIG. 5, the description thereof will be omitted.

By performing light irradiation a plurality of times, followed by acoustic wave reception and image reconstruction, time-series three-dimensional image data corresponding to the plurality of times of light irradiation are generated. Photoacoustic image data and spectral image data can be used as the three-dimensional image data. The photoacoustic image data here refer to image data showing the distribution of absorption coefficient and the like, and the spectral image data refer to image data indicating the concentration and the like that are generated on the basis of the photoacoustic image data corresponding to each wavelength when the subject is irradiated with light of a plurality of wavelengths.

(S2100: Step of Displaying Spectroscopic Image)

The image processing apparatus 1300 as a display control means causes the display device 1400 to display a spectral image so that a region corresponding to the contrast agent and other regions can be identified based on the information about the contrast agent. As the rendering method, any method such as maximum value projection method (MIP: Maximum Integrity Projection), volume rendering, and surface rendering can be adopted. Here, setting conditions such as a display region and a line-of-sight direction when rendering a three-dimensional image two-dimensionally can be arbitrarily designated according to the observation target.

Here, an example in which 797 nm and 835 nm are set in S1400 and a spectral image is generated according to the formula (1) in step S1800 will be considered. As shown in FIG. 8, when these two wavelengths are selected, the image value corresponding to the contrast agent in the spectral image generated according to the formula (1) becomes a negative value regardless of the concentration of ICG.

The display unit 160 may be able to display a moving image. For example, the image processing apparatus 1300 may be configured to generate at least one of the first photoacoustic image 2100, the second photoacoustic image 2200, and the spectral image 2300 in time series, and generate moving image data on the basis of the generated time series images and output the generated moving image data to the display unit 160. In view of the fact that the number of times the lymphatic fluid flows is relatively small, it is also preferable to display the image thereof as a still image or a time-compressed moving image in order to shorten the determination time of the user. In addition, in the moving image display, the state of lymphatic fluid flow can be repeatedly displayed. The speed of the moving image may be a predetermined speed specified in advance or a predetermined speed designated by the user.

It is also preferable to make the frame rate of the moving image variable in the display unit 160 capable of displaying the moving image. In order to make the frame rate variable, a window for the user to manually input the frame rate, a slide bar for the user to change the frame rate, and the like may be added to the GUI in FIG. 10. Here, since the lymphatic fluid flows intermittently in the lymphatic vessel, only a part of the acquired time-series volume data can be used to confirm the lymphatic flow. Therefore, where real-time display is performed when confirming the lymphatic flow, the efficiency may decrease. Therefore, by making the frame rate of the moving image displayed on the display unit 160 variable, it is possible to fast-forward the displayed moving image so that the user can check the state of the fluid in the lymphatic vessel in a short time.

The state in which the fluid flows through a lymphatic vessel is displayed on the display unit 160 as flow information in the lymphatic vessel region. A method for displaying flow information in the lymphatic vessel region is not limited to that described hereinabove. For example, the image processing apparatus 1300 as the display control means may associate the flow information in the lymphatic vessel region with the lymphatic vessel region and cause the display on the same screen of the display device 1400 by at least one method of brightness display, color display, graph display, and numerical display. Further, the image processing apparatus 1300 as the display control means may highlight at least one lymphatic vessel region.

(S2200: Step of Displaying Classification Result of Lymphatic Vessel)

In S2200, the image processing apparatus 1300 as a state estimation means analyzes image data, automatically extracts a region of lymphatic vessels, and classifies the lymphatic vessels. The image processing apparatus 1300 as a display control means causes the display device 1400 to display the classification result of the lymphatic vessels.

The image processing apparatus 1300 as a state estimation means extracts a region of lymphatic vessels in a subject by performing image analysis of the spectral image generated in S1800. In the spectral image, for example, since it is possible to distinguish the lymphatic vessels and veins in the subject from the calculated value of the formula (1), the image processing apparatus 1300 extracts the lymphatic vessel region in the subject.

The image processing apparatus 1300 as a state estimation means classifies the extracted lymphatic vessels by analyzing the spectral image. For example, the image processing apparatus 1300 may divide a lymphatic vessel into a plurality of divided regions and classify each divided region by determining a state such as Shooting Star, contraction, congestion, retention, and DBF (Dermal backflow). Shooting Star is a healthy state in which a lymphatic fluid flows like a meteor. Contraction is a state in which the width of a specific part of a lymphatic vessel changes, pumping out lymphatic fluid (liquid). Congestion is a state in which there is a time slot when the lymphatic flow is not seen. Retention is a condition in which the lymphatic fluid hardly flows.

DBF is a state in which the lymphatic fluid is flowing back toward the skin. DBF is also inclusive of a state of interstitial leakage and lymphatic dilation. Interstitial leakage is a state in which the lymphatic fluid flows back and leaks into the interstitium. Lymphatic dilatation is a state in which refluxing lymphatic fluid remains in the dilated capillary lymphatic vessels and pre-aggregated lymphatic vessels.

The image processing apparatus 1300 may classify the lymphatic vessels not only based on the state thereof, but also based on the abundance of lymphatic vessels per unit area, the abundance ratio of lymphatic vessels per unit area, or the abundance ratio of lymphatic vessels per unit volume. The abundance of lymphatic vessels per unit area, the abundance ratio of lymphatic vessels per unit area, and the abundance ratio of lymphatic vessels per unit volume are hereinafter also referred to as the abundance, area ratio and volume ratio of lymphatic vessels. Further, the image processing apparatus 1300 may classify the lymphatic vessels on the basis of distance between the lymphatic vessels and the veins or the depth from the skin of the subject.

The lymphatic vessel region may be automatically classified as described above, or may be manually classified. When manually classified, the image processing apparatus 1300 as a specifying means can specify a part of the lymphatic vessel region and classify the specified region according to the user's instruction.

The image processing apparatus 1300 as a display control means causes the display device 1400 to display the classification result of lymphatic vessels. For example, the image processing apparatus 1300 may display the lymphatic vessel region by the hue corresponding to the state of each divided region. Further, the image processing apparatus 1300 may display the abundance, area ratio, or the volume ratio of lymphatic vessels for each unit area in the subject so that the user can confirm it. The image processing apparatus 1300 may display the distance between the lymphatic vessels and the veins and the depth of the lymphatic vessels and veins from the skin.

The image processing apparatus 1300 as a storage control means stores the classification result of lymphatic vessels in the storage device 1200 in association with the analyzed image data and patient information. When the image processing apparatus 1300 causes the display device 1400 to display the image data or the patient information, the classification result of the corresponding lymphatic vessels can be acquired from the storage device 1200 and displayed together with the image data.

At least one of the image processing apparatus 1300 and the computer 150 as an information processing device functions as a device having at least one of a spectral image acquisition means, a region determination means, a photoacoustic image acquisition means, a state estimation means, a specifying means, a display control means, and a storage control means. The means may be composed of hardware different from each other or may be composed of one hardware. Further, a plurality of means may be configured of one hardware.

In the present embodiment, the blood vessel and the contrast agent were identified by selecting a wavelength at which the image value corresponding to the contrast agent becomes negative, but the image value corresponding to the contrast agent may be any value as long as the image value corresponding to the contrast agent makes it possible to identify the blood vessel and the contrast agent. For example, the image processing described in this step can be applied even when the image value of the spectral image (oxygen saturation degree image) corresponding to the contrast agent is smaller than 60% or larger than 100%.

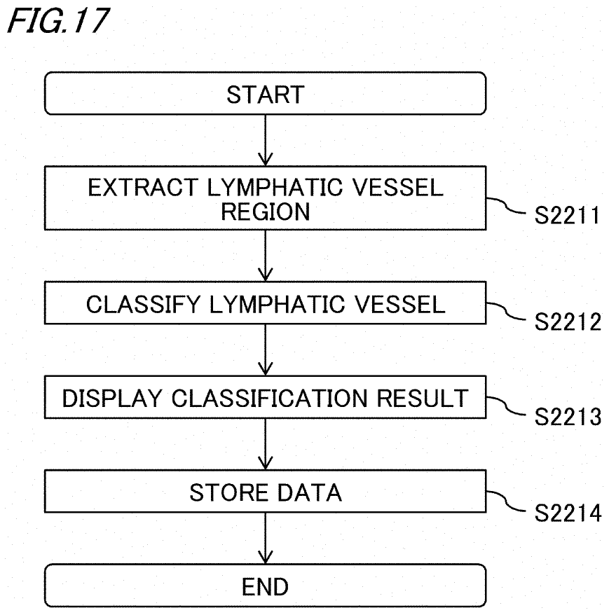

Here, the details of the process of displaying the classification result of lymphatic vessels will be described using the flowchart shown in FIG. 17.

(S2211: Step of Extracting Lymphatic Vessel Region)

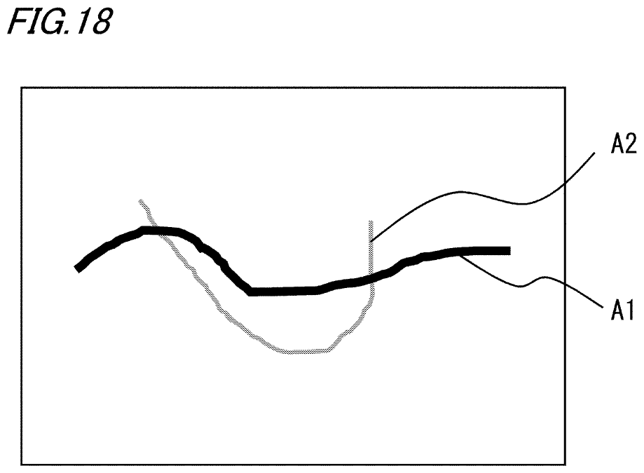

The image processing apparatus 1300 as a state estimation means extracts a lymphatic vessel region from image data. The image data for extracting the lymphatic vessel region can be, for example, a spectral image generated by using a plurality of photoacoustic images corresponding to a plurality of wavelengths. FIG. 18 is a diagram illustrating a spectral image of a subject. A method for acquiring the spectral image shown in FIG. 18 will be described hereinbelow. In the spectral image illustrated in FIG. 18, both a lymphatic vessel A1 into which the contrast agent has been injected and a vein A2 are imaged. The lymphatic vessel A1 and the vein A2 can be made distinguishable and visible by assigning at least one of hue, lightness, and chroma corresponding to their respective image values. Therefore, the image processing apparatus 1300 can extract the lymphatic vessel region by image analysis. The image data for extracting the lymphatic vessel region may be a photoacoustic image derived from a single wavelength. A lymphatic vessel can be imaged even in a photoacoustic image derived from a single wavelength, and the image processing apparatus 1300 can extract the lymphatic vessel region by image analysis. An example of a method for extracting a lymphatic vessel using a photoacoustic image derived from a single wavelength will be described hereinbelow. It is conceivable that of the images including the image data group corresponding to each of the plurality of times of light irradiation, a region where the image value changes significantly in the photoacoustic image within a predetermined period reflects the above-mentioned intermittent flow of lymphatic fluid, and this region can be taken as the lymphatic vessel region. In addition, whether a vessel in the photoacoustic image as a three-dimensional image is a lymphatic vessel or a blood vessel can be identified by storing the reference values of image values derived from hemoglobin and the contrast agent according to the depth and the thickness of structure in the computer 150 in advance.

(S2212: Step of Classifying Lymphatic Vessels)

Lymphatic vessels are classified based on various indexes such as the state of lymphatic flow and the distance to veins. By confirming the classification result, the user can specify a lymphatic vessel to be anastomosed in the anastomotic surgery that connects the lymphatic vessels and veins. Methods for classifying lymphatic vessels are illustrated below.

Lymphatic Vessel Classification Method 1

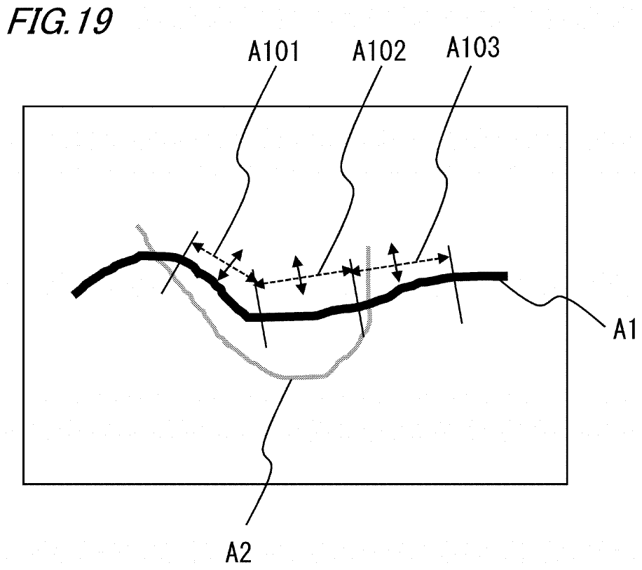

A method for classifying a lymphatic vessel by using the state of the lymphatic vessel as an index will be described with reference to FIG. 19. Here, an example is shown in which the state of a lymphatic vessel is determined based on the change of the brightness value with time. FIG. 19 shows the lymphatic vessel A1 and the vein A2. The image processing apparatus 1300 divides the lymphatic vessel A1 into regions of a predetermined length and extracts the divided regions A101, A102, and A103. The divided regions A101, A102, and A103 are approximated by, for example, a Hessian matrix, a gradient vector, or a Hough transform, and a long axis direction and a short axis direction of each region are determined.

For example, it can be determined that, among the divided regions, a divided region in which a portion having a higher brightness value moves in the long axis direction with time is in a Shooting Star state. Further, it can be determined that a divided region in which a portion having a higher brightness value becomes narrower or wider in the short axis direction is in a state of contraction. A divided region having a time slot in which the brightness value does not change can be determined to be in a state of congestion. A divided region in which the brightness value does not change can be determined to be in a state of retention.

When a divided region is in the state of DBF, whether it is interstitial leakage or lymphatic dilatation can be determined by, for example, the spatial frequency of the image. When the spatial frequency of the image is lower than a threshold value, it can be determined that the state is interstitial leakage, and when the spatial frequency is higher than the threshold value, it can be determined that the state is lymphatic dilatation.

In this way, a lymphatic vessel can be classified using the state thereof as an index. The user can determine the health of a lymphatic vessel based on the state of the lymphatic vessel, select the lymphatic vessel to be anastomosed, and determine the anastomotic position.

In this example, the change of the brightness value with time in the image was used, but the state of the lymphatic vessel may be determined not only by the brightness value, but also based on the information corresponding to the image value such as hue, lightness, and chroma described above. That is, in this example, it can be said that the state of each divided region is determined based on the change of the image value with time in each divided region.

Lymphatic Vessel Classification Method 2

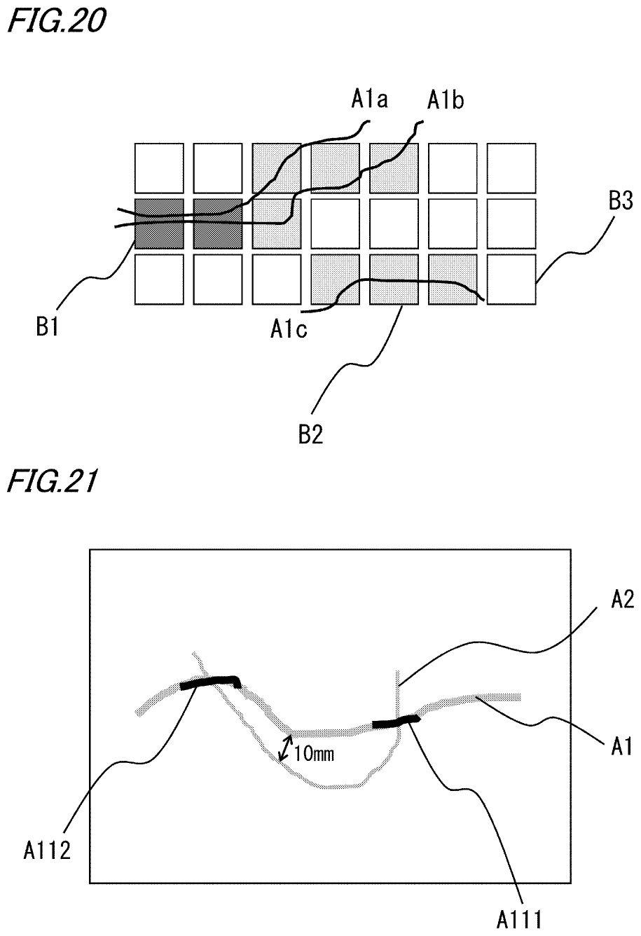

A method for classifying lymphatic vessels by using the abundance of lymphatic vessels per unit area, the area ratio, and the volume ratio of lymphatic vessels as indexes will be described with reference to FIG. 20. FIG. 20 shows three lymphatic vessels, namely, a lymphatic vessel A1a, a lymphatic vessel A1b, and a lymphatic vessel A1c. Each square block shown in FIG. 20 indicates a region corresponding to a unit area. By analyzing the image data, the image processing apparatus 1300 calculates the abundance of lymphatic vessels and the area ratio to the unit area for each unit area (for example, 2 cm²) of the subject. When the image data is an image representing a three-dimensional spatial distribution, the image processing apparatus 1300 can calculate the volume ratio of the lymphatic vessels (occupying the unit volume) to the unit area.

In the example shown in FIG. 20, each block is color-coded according to the abundance of lymphatic vessels. That is, a block B1 having two lymphatic vessels, a block B2 having one lymphatic vessel, and a block B3 having no lymphatic vessel are shown in different colors. The image processing apparatus 1300 may display each block in different colors not only according to the abundance of lymphatic vessels per unit area, but also according to the area ratio of the lymphatic vessels to the unit area, or the volume ratio of the lymphatic vessels to the unit volume.

In this way, lymphatic vessels can be classified using the abundance of lymphatic vessels per unit area and the area ratio and volume ratio of the lymphatic vessels as indexes. The user can select the lymphatic vessel to be anastomosed and determine the anastomotic position in consideration of the abundance of lymphatic vessels, the area ratio, and the volume ratio.

Lymphatic Vessel Classification Method 3

A method for classifying lymphatic vessels by using the distance between a lymphatic vessel and a vein as an index will be described with reference to FIG. 21. The image processing apparatus 1300 extracts the lymphatic vessel A1 and the vein A2 displayed in the image data and calculates the distance therebetween. The image processing apparatus 1300 can display the distance between the lymphatic vessel A1 and the vein A2 as shown in FIG. 21. The distance between the lymphatic vessel A1 and the vein A2 may be the distance in the image representing a two-dimensional spatial distribution or the distance in the image representing a three-dimensional spatial distribution. A position for displaying the distance between the lymphatic vessel A1 and the vein A2 may be designated by the user. Further, the distance between the lymphatic vessel A1 and the vein A2 may be displayed at a predetermined interval along the lymphatic vessel A1. In this case, the image processing apparatus 1300 may not display the distance at a position where the distance between the lymphatic vessel A1 and the vein A2 exceeds a predetermined threshold value.

Further, the image processing apparatus 1300 may highlight the positions where the lymphatic vessel A1 and the vein A2 intersect in a plan view (A111 and A112 in FIG. 21). Further, the position where the distance between the lymphatic vessel A1 and the vein A2 calculated in the three-dimensional image data is short may be highlighted. In addition, the lymphatic vessel A1 and the vein A2 may be displayed by assigning brightness according to the depth from the skin. In this way, lymphatic vessels can be classified using the distance to veins and the depth from the skin as indexes. The user can select the lymphatic vessels to be anastomosed and determine the anastomotic position on the basis of distance between the lymphatic vessel and the vein or the depth from the skin. The user can select each of the above indexes according to the position of the region of interest. The image processing apparatus 1300 can display the region of the lymphatic vessel classified by the selected index on the display device 1400. Further, the position where the distance between the lymphatic vessel and the vein calculated in the three-dimensional image data is short may be highlighted.

(S2213: Step of Displaying Classification Result)

When the image processing apparatus 1300 as a display control means performs the classification by using the state of lymphatic vessels as an index (lymphatic vessel classification method 1), each divided region of the lymphatic vessel can be displayed in a hue corresponding to the state. When the image processing apparatus 1300 performs the classification by using the abundance of lymphatic vessels per unit area, the area ratio, and the volume ratio as indexes (lymphatic vessel classification method 2), each block indicating the unit area may be displayed in a hue according to the value of the abundance, area ratio, and volume ratio of lymphatic vessels. When the image processing apparatus 1300 performs the classification by using the distance between the lymphatic vessel and the vein as an index (lymphatic vessel classification method 3), the image processing apparatus 1300 may not only display the distance between the lymphatic vessel and the vein, but also may assign and display at least one of brightness value, hue, lightness, and chroma according to the depth from the skin. At this time, from the viewpoint of visibility, it is preferable that the index assigned to the information related to the depth from the skin be an index that can be distinguished from other information. For example, when assigning a hue to information indicating the state of lymphatic vessels, an index other than hue is assigned to information related to the depth from the skin. That is, at least one of the brightness value, hue, lightness, and chroma corresponding to the state of the lymphatic vessel is assigned to the image value of the lymphatic vessel region. Also, at least one of brightness value, hue, lightness, and chroma, excluding those assigned to the state of the lymphatic vessel, is assigned to information related to the depth from the skin of the subject.

In addition, the image processing apparatus 1300 may evaluate indexes such as the state of lymphatic vessels, the abundance of lymphatic vessels per unit area, the distance to veins, and the depth from the skin, and highlight lymphatic vessels and veins suitable for anastomosis. A lymphatic vessel suitable for anastomosis is preferably a lymphatic vessel in which lymphatic fluid flows and is in a healthy state (for example, in a Shooting Star state), the distance from a vein is shorter, and the depth from the skin is smaller. The image processing apparatus 1300 can specify a lymphatic vessel for which indexes such as the state of lymphatic vessel, the abundance and the like per unit area, the distance to veins, and the depth from the skin satisfy predetermined conditions as a lymphatic vessel suitable for anastomosis. The image processing apparatus 1300 may further evaluate whether a lymphatic vessel is suitable for anastomosis on the basis of a state in the regions upstream and downstream of the lymphatic vessel. By highlighting the lymphatic vessel that is suitable for anastomosis, the user can select the lymphatic vessel that is more suitable for anastomosis.

(S2214: Step of Storing Data)

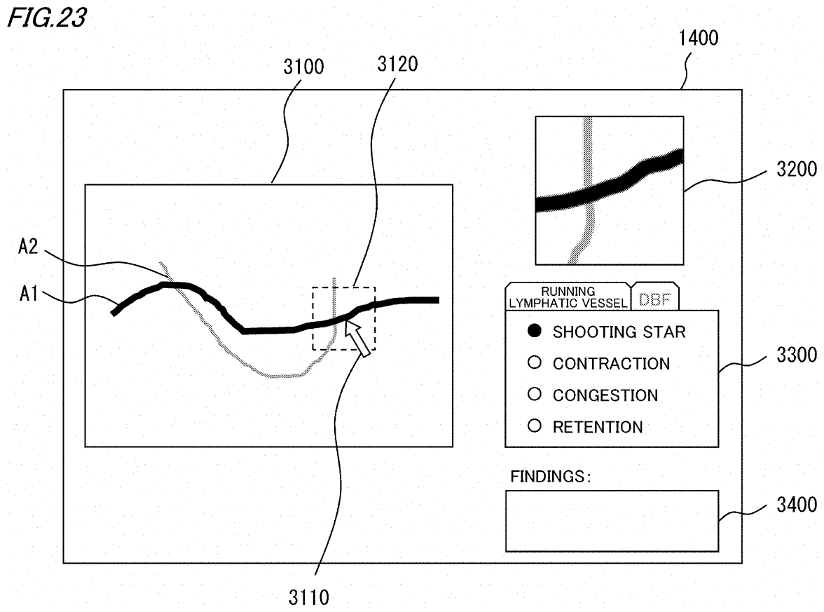

The image processing apparatus 1300 as a storage control means may store the classification result of lymphatic vessels obtained in S2212 in the storage device 1200 in association with the analyzed image data and patient information. In this case, the image processing apparatus 1300 can display the classification result of lymphatic vessels stored in the storage device 1200 on the display device 1400 together with the image data. The image processing apparatus 1300 can display the classification result of lymphatic vessels in a mode (for example, FIGS. 20 and 21) corresponding to an index selected by the user. The user can repeatedly check the classification result of lymphatic vessels associated with the patient information. The patient information may include information on physicochemical therapy given to the patient in addition to the above-mentioned patient ID. This makes it easier for the user to ascertain changes in the state of the lymphatic vessels associated with physicochemical therapy. Further, an interface that allows the user to select which mode to adopt may be added on the GUI shown in FIGS. 10 and 23.

(Method for Acquiring Spectroscopic Image)

A method for acquiring the spectral image shown in FIG. 18 (first acquisition method) will be described hereinbelow.

Since the region where the contrast agent injected into the body is present can be visualized by a spectral image, the lymphatic vessel into which the contrast agent has been injected can be visualized. However, the position of the lymphatic vessel may not be shown correctly with only one image. This is because the flow of lymphatic fluid is not as constant as blood.

Blood is constantly circulated by the beating of the heart, but the lymphatic vessels do not have a common organ that acts as a pump, and the lymphatic fluid is transported by contraction of smooth muscles present in the lymphatic vessel walls that constitute a lymphatic vessels. In addition to the contraction of the smooth muscles of the lymphatic vessel wall that occurs once every few tens of seconds to several minutes, the lymphatic fluid moves due muscle contraction that occurs with human movement, pressure caused by relaxation, a pressure change caused by breathing, and external massage stimulation. Therefore, the movement timing of the lymphatic fluid is not constant, and the lymphatic fluid flows intermittently at irregular intervals such as once every several tens of seconds to several minutes. Even if a spectral image is acquired when the lymphatic fluid is not moving, there is a concern that because a sufficient amount of contrast agent is not present in the lymphatic vessel, the lymphatic vessel cannot be visualized, or only a part of the lymphatic vessel can be visualized. That is, with an image of only one frame in the moving image, a state can be obtained in which only a portion of the lymphatic vessel in which the contrast agent is present is visualized.

Therefore, in the system according to the present embodiment, a plurality of spectral images (a plurality of first image data) along the time series is acquired in a predetermined period, and a region in which a lymphatic vessel is present (that is, the area through which the contrast agent passes) is extracted based on the acquired plurality of spectral images. In the present embodiment, the photoacoustic device 1100 acquires a plurality of spectral images along the time series in the processes of steps S1500 to S1800 and stores the acquired spectral images in the storage device 1200. The predetermined period is preferably longer than the cycle in which the movement of lymphatic fluid occurs (for example, longer than about 40 sec to 2 min).

Step S1800 is a step of generating a moving image on the basis of the plurality of spectral images.

By displaying the plurality of spectral images as moving images, the user of the device can observe how the lymphatic fluid moves. However, since the lymphatic fluid flows intermittently in the lymphatic vessels, only some spectral images among the plurality of spectral images acquired in time series can be used to confirm the flow of lymphatic fluid. That is, when the spectral image is displayed only by the moving image, the user must keep looking at the screen until the movement of the lymphatic fluid occurs. Further, since the movement cycle of the lymphatic fluid (contrast agent) takes a short time, it is difficult for the user to accurately ascertain the position of the lymphatic vessel on the screen.

Accordingly, in the present embodiment, after executing step S1800, the image processing apparatus 1300 generates a still image (second image data) showing the position of the lymphatic vessel on the basis of the plurality of spectral images. The spectral image of FIG. 18 shows the positions of the lymphatic vessel specified in this way.

Next, after the processing of step S1800 is completed, the image processing apparatus 1300 acquires a plurality of spectral images (spectral images of a plurality of frames) stored in the storage device 1200, and generates an image representing a region in which a lymphatic vessel is present.

In this step, first, a region in which the image value is within a predetermined range is extracted for each of a plurality of spectral images obtained in time series. In the above-described example, a set of pixels for which the image value, which is the calculated value of the formula (1), is a negative value is extracted. As a result, as shown in FIG. 11A, a region (shown by a black line) is extracted for each frame of the moving image, that is, for each spectral image constituting the moving image. The extracted region is the region where the contrast agent is present in each frame. Although a two-dimensional image is illustrated in FIG. 11, when the spectral image is a three-dimensional spectral image, a region may be extracted from the three-dimensional space.

Then, the regions obtained for each frame are superimposed (combined) to generate a region corresponding to the lymphatic vessel. By superimposing the regions shown in FIG. 11A, the region corresponding to the lymphatic vessel (reference numeral 1101) as shown in FIG. 11B is obtained.

The image processing apparatus 1300 generates and outputs an image (second image data) representing the position of the lymphatic vessel on the basis of the region generated in this way. When generating an image showing the position of a lymphatic vessel, a hue corresponding to the original image value (that is, the image value of the spectral image) may be given, or highlighted display may be performed by applying a unique marking. Further, the brightness corresponding to the absorption coefficient may be given. The absorption coefficient can be acquired from the photoacoustic image used to generate the spectral image.

The generated image may be outputted to the same screen as the GUI shown in FIG. 10 or may be outputted to another screen. The second image may be a three-dimensional image or a two-dimensional image. Further, an interface for storing the second image data generated as described above in the image server 1210, the storage device 1200, or the like may be added to the GUI shown in FIG. 10. Since the amount of the second image data is smaller than that of the first image data which are a moving image, the position of the lymphatic vessel can be easily ascertained even when a terminal having relatively low processing capacity is used.

According to the first method for acquiring a spectral image, it becomes possible to provide a still image showing the position of a lymphatic vessel to a user such as a doctor. Since the lymphatic fluid (contrast agent) moves periodically, the position of a lymphatic vessel cannot be accurately presented when a plurality of spectral images is simply added (or averaged). Meanwhile, in the present embodiment, since the region in which the image value is in a predetermined range is extracted from each frame of the spectral image and the extracted regions are combined, the information in the time direction is compressed. This makes it possible to accurately visualize the position of the lymphatic vessel.

In the illustrated embodiment, a region in which the image value of the spectral image is within a predetermined range is extracted, but region extraction may be performed in combination with other conditions. For example, a photoacoustic image (an image representing an absorption coefficient) corresponding to a spectral image may be referred to, and a region where the brightness value thereof is below a predetermined threshold value may be excluded. This is because even if the image value of the spectral image is within a predetermined range, the region where the absorption coefficient is small is likely to be noise. Further, the threshold value of the brightness value for filtering may be changed by the user.

Further, in the present embodiment, the wavelengths of the irradiation light (two wavelengths) were selected so that the image value of the pixel corresponding to the blood vessel region becomes positive and the image value of the pixel corresponding to the contrast agent region becomes negative, but any two wavelengths may be selected so that the signs of both image values in the spectral image are reversed.

(Another Method for Acquiring Spectroscopic Image)

Another method (second acquisition method) for acquiring the spectral image shown in FIG. 18 will be described hereinbelow.

In the method described above, in the step after step S1800, the extraction processing of each region was performed with respect to each frame of the spectral image acquired in time series, and a plurality of extracted regions was combined. Meanwhile, it is also conceivable to directly extract a region satisfying the conditions within a predetermined period by referring to a plurality of frames of the spectral image acquired in time series.

In this example, in the step after step S1800, a plurality of spectral images included in a predetermined period is selected, and a region in which the image value falls within a predetermined range within the predetermined period (in the above-mentioned example, the region where the image value is negative) is extracted. It can be said that the region where the image value falls within the predetermined range within the predetermined period is the region through which the contrast agent has passed. The predetermined period is preferably longer than the cycle in which the movement of lymphatic fluid occurs (for example, longer than about 40 seconds to 2 minutes).

FIG. 12 is a diagram illustrating a change of an image value with time at a certain pixel P(x, y) in a spectral image within a predetermined period. The illustrated pixel is the extraction target because the image value is within a predetermined range.

Thus, the contrast agent region may be extracted based on the image value that changes within the predetermined period. When making the determination, a peak hold of the image value of the photoacoustic image may be performed within the predetermined period.

As a noise countermeasure, in the second acquisition method as well, the region where the absorption coefficient is lower than the predetermined value may be excluded in the same manner as in the first acquisition method. That is, a region in which the image value of the spectral image is within the predetermined range and the brightness of the corresponding photoacoustic image exceeds the threshold value may be the extraction target.

Further, as a noise countermeasure, a region where a state in which the above-mentioned conditions are satisfied is maintained for a certain period of time may be the extraction target. Further, the certain period of time may be adjustable by the user.

### Fourth Embodiment

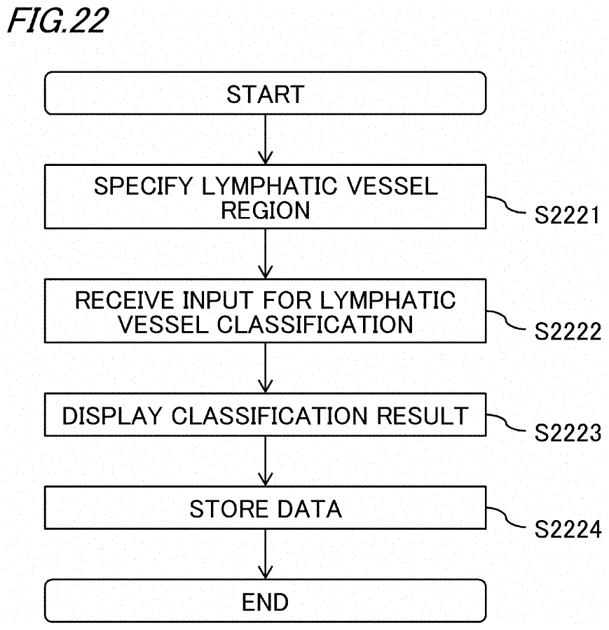

In the third embodiment, the image processing apparatus 1300 automatically classifies the lymphatic vessels and estimates the state of the lymphatic vessels by image analysis of the image data including the lymphatic vessel region. Meanwhile, in the fourth embodiment, the user specifies a part of the lymphatic vessel region in the image data including the lymphatic vessel region and determines the state of the specified region (hereinafter referred to as the region of interest). The image processing apparatus 1300 displays an input interface, which is for the user to input information such as a determination result for the region of interest, on the display device 1400. The user can input data related to the region of interest, such as the state of the region of interest and findings about the region of interest, via the input interface. The information inputted by the user is stored in the storage device 1200 in association with the image data. Further, the information inputted by the user may be stored in the storage device 1200 in association with the corresponding region of interest. Hereinafter, the image processing method according to the fourth embodiment will be described with reference to the flowchart shown in FIG. 22.

(S2221: Step of Specifying Lymphatic Vessel Region)

The image processing apparatus 1300 as a specifying means first extracts a lymphatic vessel region from the image data as in the step of S2211 in the third embodiment. The image processing apparatus 1300 specifies a part of the extracted lymphatic vessel region as a region of interest. The image processing apparatus 1300 can, for example, set a region of a predetermined length including a position specified by the user as a region of interest. By repeating the process shown in FIG. 22, the image processing apparatus 1300 can divide the lymphatic vessel region into a plurality of regions of interest and receive input of information for each region of interest.

The image processing apparatus 1300 may specify the region of interest on the basis of user's instruction. For example, in the image data displayed on the display device 1400, the user can indicate the position of the region of interest by pointing, with a pointing device such as a mouse, to the region of the lymphatic vessel region which is wished to be specified. For example, the image processing apparatus 1300 may specify a region having a predetermined length including a position designated by the user as a region of interest. Further, the image processing apparatus 1300 may specify the region of interest by causing the user to designate the positions of the start point and the end point.

A GUI for the user to indicate the position of the region of interest will be described with reference to FIG. 23. Image data to be analyzed are displayed in an item 3100. In the example shown in FIG. 23, the lymphatic vessel A1 and the vein A2 are displayed in the item 3100. The image data displayed in the item 3100 may be a moving image.

The user points with a mouse to the position wished to be specified as the region of interest. In the example shown in FIG. 23, the position pointed by the user is indicated by an arrow 3110. The image processing apparatus 1300 enlarges and displays the square area centered on the position pointed by the user, that is, a region 3120 surrounded by the dotted line in an item 3200. The lymphatic vessel region contained within the region 3120 is the specified region of interest. The size of the region of interest (the length of the specified lymphatic vessel) may be designated by the user or may be determined to a preset size by the image processing apparatus 1300. When a plurality of lymphatic vessels is included in the region of interest designated by the user, the image processing apparatus 1300 may change the region of interest to include only one of the lymphatic vessels. A moving image corresponding to the region 3120 may be displayed in the item 3200. Further, where the image shown in the item 3100 is a moving image, the user can observe the image at the same time by setting the image synchronized with the moving image in the region 3120. An item 3300 and an item 3400 will be described in the step of S2222.

(S2222: Step of Receiving Input for Lymphatic Vessel Classification)

The image processing apparatus 1300 as a display control means displays the input interface for receiving the input for the region of interest specified in S2221 on the display device 1400. The item 3300 and the item 3400 illustrated in FIG. 23 correspond to the input interface for receiving an input corresponding to the region of interest.

The item 3300 is an input interface for inputting the state of the region of interest displayed in the item 3200. The item 3300 includes tabs for “Running Lymphatic Vessel” and “DBF”. FIG. 23 shows a state in which the “Running Lymphatic Vessel” tab is selected. In the item 3300, the user can select any one of Shooting Star, contraction, stagnation, and retention as the state of the region of interest. Also, when the “DBF” tab is selected, for example, the state of interstitial leakage and lymphatic dilation is displayed as options.

The item 3400 is an input interface for inputting findings for the region of interest displayed on item 3200. The input interface is not limited to the state of the region of interest and the findings for the region of interest and may receive the input of various types of information such as the degree of suitability as an anastomosis position in lymphaticovenous anastomosis.

In addition, where a plurality of lymphatic vessels is included in the region of interest, an interface may be used that allows the state of the lymphatic vessel inputted by the user and the lymphatic vessels to be the target of the findings to be specified in the item 3100 or the item 3200. By storing the information on the specified lymphatic vessel together with the state of the lymphatic vessel and findings information, it is possible to easily ascertain which lymphatic vessel was the object of evaluation even in subsequent observation.

(S2223: Step of Displaying Classification Result)

The image processing apparatus 1300 as a display control means can display the lymphatic vessel A1 in the item 3100 by color-coding for each region of interest on the basis of the state selected in the item 3300 as the classification result of the lymphatic vessel A1.

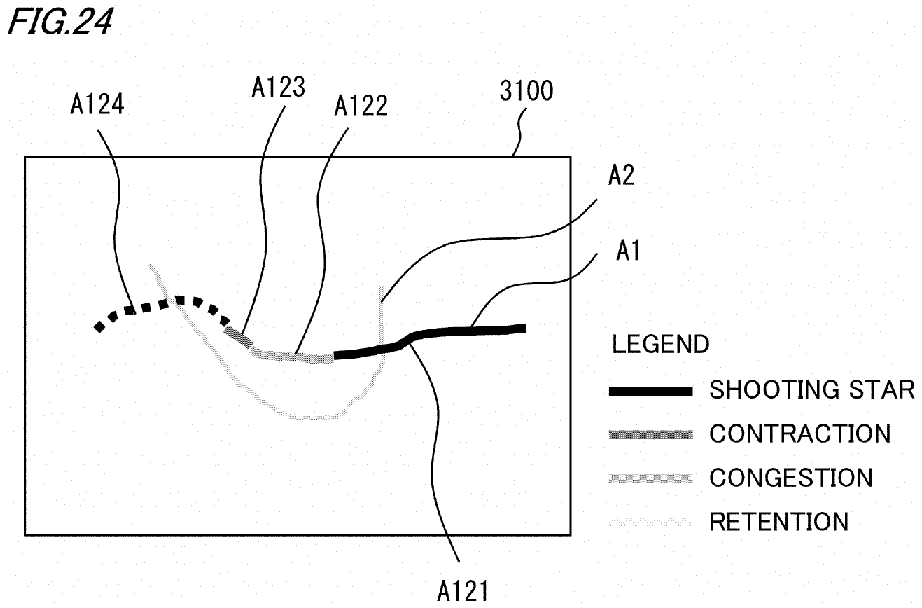

An example of displaying the classification result of lymphatic vessels according to the fourth embodiment will be described with reference to FIG. 24. FIG. 24 shows an example in which the classification result is displayed in the item 3100 of the GUI shown in FIG. 23. The lymphatic vessel A1 and the vein A2 are displayed in the item 3100. The example of FIG. 24 shows a state in which a region A121 of interest, a region A122 of interest, and a region A123 of interest are specified in the lymphatic vessel A1. A region A124 is a region of an unclassified lymphatic vessel that has not been specified as a region of interest. As shown in a legend, the region A121 of interest is in a Shooting Star state, the region A122 of interest is in a stagnation state, and the region A123 of interest is in a contraction state. The unclassified region A124 may be displayed by, for example, blinking. The image processing apparatus 1300 can prompt the user to instruct the classification of lymphatic vessels by blinking the unclassified area. A method for displaying the region specified as the region of interest and the region not specified as the region of interest in different modes is not limited to blinking display. For example, the same effect can be obtained by displaying an unclassified region in a color different from the color given to the classified region, or by displaying a frame indicating such region.

(S2224: Process of Saving Data)

The image processing apparatus 1300 may store the lymphatic vessel classification result in S2212 in the storage device 1200 in association with the analyzed image data and patient information. The image processing apparatus 1300 can display the classification result of the lymphatic vessels stored in the storage device 1200 on the display device 1400 together with the image data. When the image data is a moving image, the user can check his/her own classification result while playing back the moving image.

The flow shown in FIG. 22 illustrates a process of inputting information such as a state or a finding for one region of interest. By repeating the flow shown in FIG. 22 for the unclassified region, the lymphatic vessel region is divided into a plurality of regions of interest and classified according to each state. The step of displaying the classification result (S2223) and the step of storing the data (S2224) are executed for each region of interest in the flow shown in FIG. 22, but these steps may be executed after the input for the plurality of regions of interest is completed.

### Other Embodiments

The respective embodiments are examples used for explaining the present invention, and the present invention can be implemented by changing or combining, as appropriate, the embodiments without departing from the spirit of the present invention.

For example, the present invention can be also implemented as a photoacoustic device including at least some of the abovementioned means. Further, the present invention can also be implemented as a method for acquiring subject information including at least some of the abovementioned processes. The processes and means can be freely combined with each other for implementation unless such combinations incur technical conflicts.

For example, in the description of the embodiments, a region in which the image value of the spectral image was within a predetermined range was an extraction target, but a region in which a change in the image value (within a predetermined period) satisfies the condition may be extracted as a contrast agent region. For example, a region in which the amount of change of the image value with time exceeds the threshold value may be extracted as a contrast agent region. With such a configuration, the region can be extracted based on the pulsation of the lymph. In addition to this, a region in which the standard deviation of the image value and the fluctuation period satisfy the condition may be extracted.

According to the present invention, it is possible to provide an image processing apparatus utilized for a system that facilitates ascertaining the structure and state of a contrast-enhanced object by photoacoustic imaging and improves convenience in observing the structure of the contrast-enhanced object.

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

The present invention is not limited to the above embodiments, and various changes and modifications can be made without departing from the spirit and scope of the present invention. Accordingly, the following claims are attached to clarify the scope of the present invention public.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

What is claimed is:

1. An image processing apparatus comprising at least one memory and at least one processor which function as:
a data acquisition unit configured to acquire, in time series, first image data that is generated based on acoustic waves generated by irradiating a subject, into which a contrast agent has been injected, with light a plurality of times and that correspond respectively to the plurality of times of light irradiation; and
an image generation unit configured to generate second image data indicating a region corresponding to the contrast agent in the plurality of first image data on the basis of the plurality of the first image data acquired in time series.

2. The image processing apparatus according to claim 1, wherein the image generation unit is configured to extract a region, in which an image value is in a predetermined range, for each of the plurality of first image data and takes a region, obtained by combining a plurality of extracted regions, as the region corresponding to the contrast agent.

3. The image processing apparatus according to claim 1, wherein the image generation unit is configured to extract a region, in which an image value is in a predetermined range, in the plurality of first image data included in a predetermined period among the first image data acquired in the time series and takes the extracted region as the region corresponding to the contrast agent.

4. The image processing apparatus according to claim 1, wherein
the data acquisition unit is further configured to acquire an absorption coefficient distribution in the subject, and
the image generation unit is configured to exclude a region, in which the absorption coefficient does not exceed a predetermined threshold value, from the region corresponding to the contrast agent.

5. The image processing apparatus according to claim 1, wherein the first image data are a spectral image generated by performing computations on a plurality of photoacoustic image data obtained by irradiating the subject with light of a plurality of wavelengths different from each other.

6. The image processing apparatus according to claim 5, wherein the plurality of wavelengths are 797 nm and 835 nm.

7. An image processing method comprising:
acquiring, in time series, first image data that is generated based on acoustic waves generated by irradiating a subject, into which a contrast agent has been injected, with light a plurality of times and that correspond respectively to the plurality of times of light irradiation; and
generating second image data indicating a region corresponding to the contrast agent in the plurality of first image data on the basis of the plurality of the first image data acquired in time series.

8. A non-transitory computer-readable medium storing a program for causing a computer to execute the image processing method according to claim 7.

9. An image processing apparatus processing image data generated based on photoacoustic waves generated from inside a subject by irradiating the subject with light, the image processing apparatus comprising at least one memory and at least one processor which function as:
a state estimation unit configured to estimate a state of a lymphatic vessel by image analysis of the image data including a region of the lymphatic vessel in the subject.

10. The image processing apparatus according to claim 9, wherein the state estimation unit is configured to estimate the state of the lymphatic vessel by dividing the region of the lymphatic vessel into a plurality of divided regions and determining a state of each of the divided regions on the basis of a change of an image value with time in each of the divided regions.

11. The image processing apparatus according to claim 9, wherein the state estimation unit is configured to estimate the state of the lymphatic vessel by calculating at least any of abundance of lymphatic vessels per unit area, an abundance ratio of the lymphatic vessels per unit area, and an abundance ratio of the lymphatic vessels per unit volume in the image data.

12. The image processing apparatus according to claim 9, wherein the state estimation unit is configured to estimate the state of the lymphatic vessel by calculating a distance between a vein and the lymphatic vessel in the image data including the vein and the region of the lymphatic vessel in the subject.

13. The image processing apparatus according to claim 9, wherein the image data are time-series three-dimensional image data that is generated based on photoacoustic waves generated by irradiating the subject with light a plurality of times and that include images corresponding respectively to the plurality of times of light irradiation.

14. An image processing method comprising:
generating image data on the basis of photoacoustic waves generated from inside a subject by irradiating the subject with light; and
estimating a state of a lymphatic vessel by image analysis of the image data including a region of a lymphatic vessel in the subject.

15. A non-transitory computer-readable medium storing a program for causing a computer to execute the image processing method according to claim 14.

16. An image processing apparatus processing image data generated based on photoacoustic waves generated from inside a subject by irradiating the subject with light, the image processing apparatus comprising at least one memory and at least one processor which function as:
a display control unit configured to display the image data and an input interface that receives an input related to a region of interest, which is a part of a region of a lymphatic vessel in the subject in the image data, on a display device; and
a storage control unit configured to store the image data in association with information inputted via the input interface in a storage device.

17. The image processing apparatus according to claim 16, wherein the at least one memory and the at least one processor further function as:
a specifying unit configured to specify a part of the region of the lymphatic vessel as the region of interest by image analysis of the image data including the region of the lymphatic vessel in the subject.

18. The image processing apparatus according to claim 16, wherein the display control unit is configured to cause the display device to display information, which is stored in the storage device, in association with the image data displayed on the display device.

19. The image processing apparatus according to claim 16, wherein the image data are time-series three-dimensional image data that is generated based on photoacoustic waves generated by irradiating the subject with light a plurality of times and that include images corresponding respectively to the plurality of times of light irradiation.

20. An image processing method comprising:
generating image data on the basis of photoacoustic waves inside a subject by irradiating the subject with light;
causing a display device to display the image data and an input interface that receives an input related to a region of interest, which is a part of a region of a lymphatic vessel in the subject in the image data; and
storing the image data in association with information inputted via the input interface in a storage device.

21. A non-transitory computer-readable medium storing a program for causing a computer to execute the image processing method according to claim 20.
